(19) United States
(12) Patent Application Publication
(10) Pub. No.: US 2007/0161683 A1
(43) Pub. Date: Jul. 12, 2007

(54) Certain chemical entities, compositions, and methods

(76) Inventors: Scott Collibee, San Carlos, CA (US); Gustave Bergnes, Pacifica, CA (US); Matthew R. Hamilton, San Francisco, CA (US); Bradley P. Morgan, Moraga, CA (US); David J. Morgans JR., Los Altos, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/600,014

(22) Filed: Nov. 14, 2006

Related U.S. Application Data
(60) Provisional application No. 60/737,107, filed on Nov. 15, 2005.

(51) Int. Cl. A61K 31/4439; C07D 401/02

(52) U.S. Cl. 514/341; 514/355; 546/268.1; 546/315

(57) ABSTRACT

Compounds useful for treating cellular proliferative diseases are disclosed.

This application claims the benefit of provisional U.S. Patent Application No. 60/737,107, filed Nov. 15, 2005, which is hereby incorporated by reference.

Provided are certain chemical entities that cause mitotic arrest and cell death and are useful in the treatment of cellular proliferative diseases, for example cancer.

Improvements in the specificity of agents used to treat cancer is of considerable interest. Reducing the side effects associated with the administration of these agents would result in significant therapeutic benefits. Traditionally, dramatic improvements in the treatment of cancer have been associated with identification of therapeutic agents acting through novel mechanisms. Examples of such agents include not only the taxanes, but also the camptothecin class of topoisomerase I inhibitors.

Provided is at least one chemical entity chosen from compounds of Formula I: 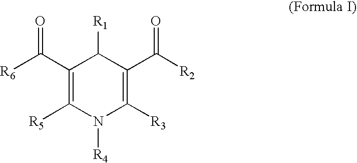
and pharmaceutically acceptable salts, solvates, chelates, non-covalent complexes, prodrugs, and mixtures thereof, wherein
R₁ is chosen from optionally substituted cycloalkyl, optionally substituted heterocycloalkyl, optionally substituted aryl and optionally substituted heteroaryl;
R₂ is chosen from optionally substituted aryl, optionally substituted heteroaryl, optionally substituted alkoxy, and optionally substituted amino;
R₃ is chosen from hydrogen and optionally substituted alkyl;
R₄ is chosen from hydrogen, optionally substituted alkyl, optionally substituted heterocycloalkyl, optionally substituted acyl, optionally substituted aryl, optionally substituted heteroaryl, aminocarbonyl, sulfonyl, optionally substituted alkoxycarbonyl, and optionally substituted cycloalkyl;
R₅ is chosen from hydrogen and optionally substituted alkyl; and
R₆ is chosen from optionally substituted aryl, optionally substituted heteroaryl, optionally substituted alkoxy, and optionally substituted amino.

Also provided is a pharmaceutical composition comprising a therapeutically effective amount of at least one chemical entity described herein together with at least one pharmaceutically acceptable vehicle chosen from carriers, adjuvants, and excipients.

Also provided is a packaged pharmaceutical composition, comprising
a pharmaceutical composition described herein; and
instructions for using the composition to treat a patient suffering from a cellular proliferative disease.

Also provided is a method for treating a patient having a cellular proliferative disease, comprising administering to the patient an effective amount of at least one chemical entity of described herein.

As used herein, when any variable occurs more than one time in a chemical formula, its definition on each occurrence is independent of its definition at every other occurrence.

A dash (“—”) that is not between two letters or symbols is used to indicate a point of attachment for a substituent. For example, —CONH₂ is attached through the carbon atom.

By “optional” or “optionally” is meant that the subsequently described event or circumstance may or may not occur, and that the description includes instances where the event or circumstance occurs and instances in which it does not. For example, “optionally substituted alkyl” encompasses both “alkyl” and “substituted alkyl” as defined below. It will be understood by those skilled in the art, with respect to any group containing one or more substituents, that such groups are not intended to introduce any substitution or substitution patterns that are sterically impractical, synthetically non-feasible and/or inherently unstable.

“Alkyl” encompasses straight chain and branched chain having the indicated number of carbon atoms, usually from 1 to 20 carbon atoms, for example 1 to 8 carbon atoms, such as 1 to 6 carbon atoms. For example C₁-C₆ alkyl encompasses both straight and branched chain alkyl of from 1 to 6 carbon atoms. Examples of alkyl groups include methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, and the like. Alkylene is another subset of alkyl, referring to the same residues as alkyl, but having two points of attachment. Alkylene groups will usually have from 2 to 20 carbon atoms, for example 2 to 8 carbon atoms, such as from 2 to 6 carbon atoms. For example, C₀ alkylene indicates a covalent bond and C₁ alkylene is a methylene group. When an alkyl residue having a specific number of carbons is named, all branched and straight chain versions having that number of carbons are intended to be encompassed; thus, for example, “butyl” is meant to include n-butyl, sec-butyl, isobutyl and t-butyl; “propyl” includes n-propyl and isopropyl. “Lower alkyl” refers to alkyl groups having one to four carbons.

“Alkenyl” refers to an unsaturated branched or straight-chain alkyl group having at least one carbon-carbon double bond derived by the removal of one hydrogen atom from a single carbon atom of a parent alkene. The group may be in either the cis or trans configuration about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl; and the like. In certain embodiments, an alkenyl group has from 2 to 20 carbon atoms and in other embodiments, from 2 to 6 carbon atoms.

“Alkynyl” refers to an unsaturated branched or straight-chain alkyl group having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl; butynyls such as but-1-yn-1-yl, but-1-yn-3-yl, but-3-yn-1-yl; and the like. In certain embodiments, an alkynyl group has from 2 to 20 carbon atoms and in other embodiments, from 3 to 6 carbon atoms.

“Cycloalkyl” indicates a non-aromatic carbocyclic ring, usually having from 3 to 7 ring carbon atoms. The ring may be saturated or have one or more carbon-carbon double bonds. Examples of cycloalkyl groups include cyclopropyl, cyclobutyl, cyclopentyl, cyclopentenyl, cyclohexyl, and cyclohexenyl, as well as bridged and caged saturated ring groups such as norbornane.

By “alkoxy” is meant an alkyl group of the indicated number of carbon atoms attached through an oxygen bridge such as, for example, methoxy, ethoxy, propoxy, isopropoxy, n-butoxy, sec-butoxy, tert-butoxy, pentyloxy, 2-pentyloxy, isopentyloxy, neopentyloxy, hexyloxy, 2-hexyloxy, 3-hexyloxy, 3-methylpentyloxy, and the like. Alkoxy groups will usually have from 1 to 7 carbon atoms attached through the oxygen bridge. “Lower alkoxy” refers to alkoxy groups having one to four carbons.

“Mono- and di-alkylcarboxamide” encompasses a group of the formula —(C═O)NRₐR_{b} where Rₐ and R_{b} are independently chosen from hydrogen and alkyl groups of the indicated number of carbon atoms, provided that Rₐ and R_{b} are not both hydrogen.

“Acyl” refers to the groups (alkyl)-C(O)—; (cycloalkyl)-C(O)—; (aryl)-C(O)—; (heteroaryl)-C(O)—; and (heterocycloalkyl)-C(O)—, wherein the group is attached to the parent structure through the carbonyl functionality and wherein alkyl, cycloalkyl, aryl, heteroaryl, and heterocycloalkyl are as described herein. Acyl groups have the indicated number of carbon atoms, with the carbon of the keto group being included in the numbered carbon atoms. For example a C₂ acyl group is an acetyl group having the formula CH₃(C═O)—.

By “alkoxycarbonyl” is meant a group of the formula (alkoxy)(C═O)— attached through the carbonyl carbon wherein the alkoxy group has the indicated number of carbon atoms. Thus a C₁-C₆ alkoxycarbonyl group is an alkoxy group having from 1 to 6 carbon atoms attached through its oxygen to a carbonyl linker.

By “amino” is meant the group —NH₂.

“Mono- and di-(alkyl)amino” encompasses secondary and tertiary alkyl amino groups, wherein the alkyl groups are as defined above and have the indicated number of carbon atoms. The point of attachment of the alkylamino group is on the nitrogen. Examples of mono- and di-alkylamino groups include ethylamino, dimethylamino, and methyl-propyl-amino.

The term “aminocarbonyl” refers to the group —CONR^{b}R^{c}, where
R^{b} is chosen from H, optionally substituted C₁-C₆ alkyl, optionally substituted cycloalkyl, optionally substituted heterocycloalkyl, optionally substituted aryl, and optionally substituted heteroaryl; and
R^{c} is independently chosen from hydrogen and optionally substituted C₁-C₄ alkyl; or
R^{b} and R^{c} taken together with the nitrogen to which they are bound, form an optionally substituted 5- to 7-membered nitrogen-containing heterocycloalkyl which optionally includes 1 or 2 additional heteroatoms selected from O, N, and S in the heterocycloalkyl ring;
   where each substituted group is independently substituted with one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

“Aryl” encompasses:
6-membered carbocyclic aromatic rings, for example, benzene;
bicyclic ring systems wherein at least one ring is carbocyclic and aromatic, for example, naphthalene, indane, and tetralin; and
tricyclic ring systems wherein at least one ring is carbocyclic and aromatic, for example, fluorene.
   For example, aryl includes 6-membered carbocyclic aromatic rings fused to a 5- to 7-membered heterocycloalkyl ring containing 1 or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap in any way with heteroaryl, separately defined below. Hence, if one or more carbocyclic aromatic rings is fused with a heterocycloalkyl aromatic ring, the resulting ring system is heteroaryl, not aryl, as defined herein.

The term “aryloxy” refers to the group —O-aryl.

“Carbamimidoyl” refers to the group —C(═NH)—NH₂.

“Substituted carbamimidoyl” refers to the group —C(═NR^{e})—NR^{f}R^{g} where R^{e}, is chosen from: hydrogen, cyano, optionally substituted alkyl, optionally substituted cycloalkyl, optionally substituted aryl, optionally substituted heteroaryl, and optionally substituted heterocycloalkyl; and R^{f} and R^{g} are independently chosen from: hydrogen optionally substituted alkyl, optionally substituted cycloalkyl, optionally substituted aryl, optionally substituted heteroaryl, and optionally substituted heterocycloalkyl, provided that at least one of R^{e}, R^{f}, and R^{g} is not hydrogen and wherein substituted alkyl, cycloalkyl, aryl, heterocycloalkyl, and heteroaryl refer respectively to alkyl, cycloalkyl, aryl, heterocycloalkyl, and heteroaryl wherein one or more (such as up to 5, for example, up to 3) hydrogen atoms are replaced by a substituent independently chosen from:
—R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substitutent for cycloalkyl, heterocycloalkyl, and heteroaryl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, sulfanyl (such as SR^{b}), sulfinyl (such as —SOR^{a}), and sulfonyl (such as —SO₂R^{a}and —SO₂NR^{b}R^{c}),
where R^{a}is chosen from optionally substituted C₁-C₆ alkyl, optionally substituted aryl, and optionally substituted heteroaryl;
R^{b} is chosen from H, optionally substituted C₁-C₆ alkyl, optionally substituted aryl, and optionally substituted heteroaryl; and
R^{c} is independently chosen from hydrogen and optionally substituted C₁-C₄ alkyl; or R^{b} and R^{c}, and the nitrogen to which they are attached, form an optionally substituted heterocycloalkyl group; and
where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂ NH(phenyl), —NHSO₂(C₁-C₄ alkyl), —NHSO₂(phenyl), and —NHSO₂(C₁-C₄ haloalkyl).

The term “halo” includes fluoro, chloro, bromo, and iodo, and the term “halogen” includes fluorine, chlorine, bromine, and iodine.

“Haloalkyl” indicates alkyl as defined above having the specified number of carbon atoms, substituted with 1 or more halogen atoms, up to the maximum allowable number of halogen atoms. Examples of haloalkyl include, but are not limited to, trifluoromethyl, difluoromethyl, 2-fluoroethyl, and penta-fluoroethyl.

“Heteroaryl” encompasses:
5- to 7-membered aromatic, monocyclic rings containing one or more, for example, from 1 to 4, or in certain embodiments, from 1 to 3, heteroatoms chosen from N, O, and S, with the remaining ring atoms being carbon;
bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring; and
tricyclic heterocycloalkyl rings containing one or more, for example, from 1 to 5, or in certain embodiments, from 1 to 4, heteroatoms chosen from N, O, and S, with the remaining ring atoms being carbon and wherein at least one heteroatom is present in an aromatic ring.
   For example, heteroaryl includes a 5- to 7-membered heterocycloalkyl, aromatic ring fused to a 5- to 7-membered cycloalkyl or heterocycloalkyl ring. For such fused, bicyclic heteroaryl ring systems wherein only one of the rings contains one or more heteroatoms, the point of attachment may be at either ring. When the total number of S and O atoms in the heteroaryl group exceeds 1, those heteroatoms are not adjacent to one another. In certain embodiments, the total number of S and O atoms in the heteroaryl group is not more than 2. In certain embodiments, the total number of S and O atoms in the aromatic heterocycle is not more than 1. Examples of heteroaryl groups include, but are not limited to, (as numbered from the linkage position assigned priority 1), 2-pyridyl, 3-pyridyl, 4-pyridyl, 2,3-pyrazinyl, 3,4-pyrazinyl, 2,4-pyrimidinyl, 3,5-pyrimidinyl, 2,3-pyrazolinyl, 2,4-imidazolinyl, isoxazolinyl, oxazolinyl, thiazolinyl, thiadiazolinyl, tetrazolyl, thienyl, benzothiophenyl, furanyl, benzofuranyl, benzoimidazolinyl, indolinyl, pyridazinyl, triazolyl, quinolinyl, pyrazolyl, and 5,6,7,8-tetrahydroisoquinolinyl. Bivalent radicals derived from univalent heteroaryl radicals whose names end in “-yl” by removal of one hydrogen atom from the atom with the free valence are named by adding “-idene” to the name of the corresponding univalent radical, e.g., a pyridyl group with two points of attachment is a pyridylidene. Heteroaryl does not encompass or overlap with aryl, cycloalkyl, or heterocycloalkyl, as defined herein

Substituted heteroaryl also includes ring systems substituted with one or more oxide (—O⁻) substituents, such as pyridinyl N-oxides.

By “heterocycloalkyl” is meant a single, non-aromatic ring, usually with 3 to 7 ring atoms, containing at least 2 carbon atoms in addition to 1-3 heteroatoms independently selected from oxygen, sulfur, and nitrogen, as well as combinations comprising at least one of the foregoing heteroatoms. The ring may be saturated or have one or more carbon-carbon double bonds. Suitable heterocycloalkyl groups include, for example (as numbered from the linkage position assigned priority 1), 2-pyrrolidinyl, 2,4-imidazolidinyl, 2,3-pyrazolidinyl, 2-piperidyl, 3-piperidyl, 4-piperidyl, and 2,5-piperizinyl. Morpholinyl groups are also contemplated, including 2-morpholinyl and 3-morpholinyl (numbered wherein the oxygen is assigned priority 1). Substituted heterocycloalkyl also includes ring systems substituted with one or more oxo (=0) or oxide (—O⁻) substituents, such as piperidinyl N-oxide, morpholinyl-N-oxide, 1-oxo-1-thiomorpholinyl and 1,1-dioxo-1-thiomorpholinyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteratoms independently selected from oxygen, sulfur, and nitrogen and is not aromatic.

As used herein, “modulation” refers to a change in activity as a direct or indirect response to the presence of compounds of Formula I, relative to the activity in the absence of the compound. The change may be an increase in activity or a decrease in activity, and may be due to the direct interaction of the compound with the kinesin, or due to the interaction of the compound with one or more other factors that in turn affect kinesin activity. For example, the presence of the compound may, for example, increase or decrease kinesin activity by directly binding to the kinesin, by causing (directly or indirectly) another factor to increase or decrease the kinesin activity, or by (directly or indirectly) increasing or decreasing the amount of kinesin present in the cell or organism.

The term “sulfanyl” includes the groups: —S-(optionally substituted (C₁-C₆)alkyl), —S-(optionally substituted aryl), —S-(optionally substituted heteroaryl), and —S-(optionally substituted heterocycloalkyl). Hence, sulfanyl includes the group C₁-C₆ alkylsulfanyl.

The term “sulfinyl” includes the groups: —S(O)-(optionally substituted (C₁-C₆)alkyl), —S(O)-optionally substituted aryl), —S(O)-optionally substituted heteroaryl), —S(O)-(optionally substituted heterocycloalkyl); and —S(O)-(optionally substituted amino).

The term “sulfonyl” includes the groups: —S(O₂)-(optionally substituted (C₁-C₆)alkyl), —S(O₂)-optionally substituted aryl), —S(O₂)-optionally substituted heteroaryl), —S(O₂)-(optionally substituted heterocycloalkyl), and —S(O₂)-(optionally substituted amino).

The term “substituted”, as used herein, means that any one or more hydrogens on the designated atom or group is replaced with a selection from the indicated group, provided that the designated atom's normal valence is not exceeded. When a substituent is oxo (i.e., ═O) then 2 hydrogens on the atom are replaced. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation as an agent having at least practical utility. Unless otherwise specified, substituents are named into the core structure. For example, it is to be understood that when (cycloalkyl)alkyl is listed as a possible substituent, the point of attachment of this substituent to the core structure is in the alkyl portion.

The terms “substituted” alkyl, cycloalkyl, aryl, heterocycloalkyl, and heteroaryl, unless otherwise expressly defined, refer respectively to alkyl, cycloalkyl, aryl, heterocycloalkyl, and heteroaryl wherein one or more (such as up to 5, for example, up to 3) hydrogen atoms are replaced by a substituent independently chosen from:
—R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substitutent for cycloalkyl, heterocycloalkyl, and heteroaryl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, sulfanyl (such as SR^{b}), sulfinyl (such as —SOR^{a}), and sulfonyl (such as —SO₂R^{a}and —SO₂NR^{b}R^{c}),
where R^{a} is chosen from optionally substituted C₁-C₆ alkyl, optionally substituted cycloalkyl, optionally substituted heterocycloalkyl, optionally substituted alkenyl, optionally substituted alkynyl, optionally substituted aryl, and optionally substituted heteroaryl;
R^{b} is chosen from hydrogen, optionally substituted C₁-C₆ alkyl, optionally substituted cycloalkyl, optionally substituted heterocycloalkyl, optionally substituted aryl, and optionally substituted heteroaryl; and
R^{c} is independently chosen from hydrogen and optionally substituted C₁-C₄ alkyl; or
R^{b} and R^{c}, and the nitrogen to which they are attached, form an optionally substituted heterocycloalkyl group; and
where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁C₄ alkyl), —SO2NH(phenyl), —NHSO₂(C₁-C₄ alkyl), —NHSO₂(phenyl), and —NHSO₂(C₁-C₄ haloalkyl).

The term “substituted acyl” refers to the groups (substituted alkyl)-C(O)—; (substituted cycloalkyl)-C(O)—; (substituted aryl)-C(O)—; (substituted heteroaryl)-C(O)—; and (substituted heterocycloalkyl)-C(O)—, wherein the group is attached to the parent structure through the carbonyl functionality and wherein substituted alkyl, cycloalkyl, aryl, heteroaryl, and heterocycloalkyl, refer respectively to alkyl, cycloalkyl, aryl, heteroaryl, and heterocycloalkyl wherein one or more (such as up to 5, for example, up to 3) hydrogen atoms are replaced by a substituent independently chosen from:
—R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substitutent for cycloalkyl, heterocycloalkyl, and heteroaryl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, sulfanyl (such as SR^{b}), sulfinyl (such as —SOR^{a}), and sulfonyl (such as —SO₂R^{a} and —SO₂NR^{b}R^{c}),
where R^{a} is chosen from optionally substituted C₁-C₆ alkyl, optionally substituted alkenyl, optionally substituted alkynyl, optionally substituted aryl, and optionally substituted heteroaryl;
R^{b} is chosen from H, optionally substituted C₁-C₆ alkyl, optionally substituted cycloalkyl, optionally substituted heterocycloalkyl, optionally substituted aryl, and optionally substituted heteroaryl; and
R^{c} is independently chosen from hydrogen and optionally substituted C₁-C₄ alkyl; or
R^{b} and R^{c}, and the nitrogen to which they are attached, form an optionally substituted heterocycloalkyl group; and
where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “substituted alkoxy” refers to alkoxy wherein the alkyl constituent is substituted (i.e., —O-(substituted alkyl)) wherein “substituted alkyl” refers to alkyl wherein one or more (such as up to 5, for example, up to 3) hydrogen atoms are replaced by a substituent independently chosen from:
—R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substitutent for cycloalkyl, heterocycloalkyl, and heteroaryl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, sulfanyl (such as SR^{b}), sulfinyl (such as —SOR^{a}), and sulfonyl (such as —SO₂R^{a} and —SO₂NR^{b}R^{c}),
where R^{a} is chosen from optionally substituted C₁-C₆ alkyl, optionally substituted alkenyl, optionally substituted alkynyl, optionally substituted aryl, and optionally substituted heteroaryl;
R^{b} is chosen from H, optionally substituted C₁-C₆ alkyl, optionally substituted cycloalkyl, optionally substituted heterocycloalkyl, optionally substituted aryl, and optionally substituted heteroaryl; and
R^{c} is independently chosen from hydrogen and optionally substituted C₁-C₄ alkyl; or
R^{b} and R^{c}, and the nitrogen to which they are attached, form an optionally substituted heterocycloalkyl group; and
where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl). In some embodiments, a substituted alkoxy group is “polyalkoxy” or —O-(optionally substituted alkylene)-(optionally substituted alkoxy), and includes groups such as —OCH₂CH₂OCH₃, and residues of glycol ethers such as polyethyleneglycol, and —O(CH₂CH₂O)ₓCH₃, where x is an integer of 2-20, such as 2-10, and for example, 2-5. Another substituted alkoxy group is hydroxyalkoxy or —OCH₂(CH₂)_{y}OH, where y is an integer of 1-10, such as 1-4.

The term “substituted alkoxycarbonyl” refers to the group (substituted alkyl)-O—C(O)— wherein the group is attached to the parent structure through the carbonyl functionality and wherein substituted refers to alkyl wherein one or more (such as up to 5, for example, up to 3) hydrogen atoms are replaced by a substituent independently chosen from:
—R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substitutent for cycloalkyl, heterocycloalkyl, and heteroaryl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, sulfanyl (such as SR^{b}), sulfinyl (such as —SOR^{a}), and sulfonyl (such as —SO₂R^{a} and —SO₂NR^{b}R^{c}),
where R^{a} is chosen from optionally substituted C₁-C₆ alkyl, optionally substituted alkenyl, optionally substituted alkynyl, optionally substituted aryl, and optionally substituted heteroaryl;
R^{b} is chosen from H, optionally substituted C₁-C₆ alkyl, optionally substituted cycloalkyl, optionally substituted heterocycloalkyl, optionally substituted aryl, and optionally substituted heteroaryl; and
R^{c} is independently chosen from hydrogen and optionally substituted C₁-C₄ alkyl; or
R^{b} and R^{c}, and the nitrogen to which they are attached, form an optionally substituted heterocycloalkyl group; and
where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “substituted amino” refers to the group —NHR^{d} or —NR^{d}R^{e} wherein R^{d} is chosen from: hydroxy, optionally substitued alkoxy, optionally substituted alkyl, optionally substituted cycloalkyl, optionally substituted acyl, optionally substituted carbamimidoyl, aminocarbonyl, optionally substituted aryl, optionally substituted heteroaryl, optionally substituted heterocycloalkyl, optionally substituted alkoxycarbonyl, sulfinyl and sulfonyl, and wherein R^{e} is chosen from: optionally substituted alkyl, optionally substituted cycloalkyl, optionally substituted aryl, optionally substituted heteroaryl, and optionally substituted heterocycloalkyl, and wherein substituted alkyl, cycloalkyl, aryl, heterocycloalkyl, and heteroaryl refer respectively to alkyl, cycloalkyl, aryl, heterocycloalkyl, and heteroaryl wherein one or more (such as up to 5, for example, up to 3) hydrogen atoms are replaced by a substituent independently chosen from:

—R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substitutent for cycloalkyl, heterocycloalkyl, and heteroaryl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, sulfanyl (such as SR^{b}), sulfinyl (such as —SOR^{a}), and sulfonyl (such as —SO₂R^{a} and —SO₂NR^{b}R^{c}),
where R^{a} is chosen from optionally substituted C₁-C₆ alkyl, optionally substituted alkenyl, optionally substituted alkynyl, optionally substituted aryl, and optionally substituted heteroaryl;
R^{b} is chosen from H, optionally substituted C₁-C₆ alkyl, optionally substituted cycloalkyl, optionally substituted heterocycloalkyl, optionally substituted aryl, and optionally substituted heteroaryl; and
R^{c} is independently chosen from hydrogen and optionally substituted C₁-C₄ alkyl; or
R^{b} and R^{c}, and the nitrogen to which they are attached, form an optionally substituted heterocycloalkyl group; and
where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl); and
wherein optionally substituted acyl, optionally substituted alkoxycarbonyl, sulfinyl and sulfonyl are as defined herein.

The term “substituted amino” also refers to N-oxides of the groups —NHR^{d}, and NR^{d}R^{d} each as described above. N-oxides can be prepared by treatment of the corresponding amino group with, for example, hydrogen peroxide or m-chloroperoxybenzoic acid. The person skilled in the art is familiar with reaction conditions for carrying out the N-oxidation.

Compounds of Formula I include, but are not limited to, optical isomers of compounds of Formula I, racemates, and other mixtures thereof. In those situations, the single enantiomers or diastereomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. In addition, compounds of Formula I include Z- and E- forms (or cis- and trans-forms) of compounds with carbon-carbon double bonds. Where compounds of Formula I exists in various tautomeric forms, chemical entities of the present invention include all tautomeric forms of the compound.

Chemical entities of the present invention include, but are not limited to compounds of Formula I and all pharmaceutically acceptable forms thereof. Pharmaceutically acceptable forms of the compounds recited herein include pharmaceutically acceptable salts, solvates, crystal forms (including polymorphs and clathrates), chelates, non-covalent complexes, prodrugs, and mixtures thereof. In certain embodiments, the compounds described herein are in the form of pharmaceutically acceptable salts. Hence, the terms “chemical entity” and “chemical entities” also encompass pharmaceutically acceptable salts, solvates, chelates, non-covalent complexes, prodrugs, and mixtures.

“Pharmaceutically acceptable salts” include, but are not limited to salts with inorganic acids, such as hydrochloride, phosphate, diphosphate, hydrobromide, sulfate, sulfinate, nitrate, and like salts; as well as salts with an organic acid, such as malate, maleate, fumarate, tartrate, succinate, citrate, lactate, methanesulfonate, p-toluenesulfonate, 2-hydroxyethylsulfonate, benzoate, salicylate, stearate, and alkanoate such as acetate, HOOC—(CH₂)ₙ—COOH where n is 0-4, and like salts. Similarly, pharmaceutically acceptable cations include, but are not limited to sodium, potassium, calcium, aluminum, lithium, and ammonium.

In addition, if the compound of Formula I is obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

As noted above, prodrugs also fall within the scope of chemical entities, for example ester or amide derivatives of the compounds of Formula I. The term “prodrugs” includes any compounds that become compounds of Formula I when administered to a patient, e.g., upon metabolic processing of the prodrug. Examples of prodrugs include, but are not limited to, acetate, formate, phosphate, and benzoate and like derivatives of functional groups (such as alcohol or amine groups) in the compounds of Formula I.

The term “solvate” refers to the chemical entity formed by the interaction of a solvent and a compound. Suitable solvates are pharmaceutically acceptable solvates, such as hydrates, including monohydrates and hemi-hydrates.

The term “chelate” refers to the chemical entity formed by the coordination of a compound to a metal ion at two (or more) points.

The term “non-covalent complex” refers to the chemical entity formed by the interaction of a compound and another molecule wherein a covalent bond is not formed between the compound and the molecule. For example, complexation can occur through van der Waals interactions, hydrogen bonding, and electrostatic interactions (also called ionic bonding).

The term “active agent” is used to indicate a chemical entity which has biological activity. In certain embodiments, an “active agent” is a compound having pharmaceutical utility. For example an active agent may be an anti-cancer therapeutic.

By “significant” is meant any detectable change that is statistically significant in a standard parametric test of statistical significance such as Student's T-test, where p<0.05.

The term “antimitotic” refers to a drug for inhibiting or preventing mitosis, for example, by causing metaphase arrest. Some antitumour drugs block proliferation and are considered antimitotics.

The term “therapeutically effective amount” of a chemical entity of this invention means an amount effective, when administered to a human or non-human patient, to provide a therapeutic benefit such as amelioration of symptoms, slowing of disease progression, or prevention of disease e.g., a therapeutically effective amount may be an amount sufficient to decrease the symptoms of a disease. In some embodiments, a therapeutically effective amount is an amount sufficient to reduce cancer symptoms. In some embodiments a therapeutically effective amount is an amount sufficient to decrease the number of detectable cancerous cells in an organism, detectably slow, or stop the growth of a cancerous tumor. In some embodiments, a therapeutically effective amount is an amount sufficient to shrink a cancerous tumor.

The term “inhibition” indicates a significant decrease in the baseline activity of a biological activity or process.

“Treatment” or “treating” means any treatment of a disease in a patient, including:
a) preventing the disease, that is, causing the clinical symptoms of the disease not to develop;
b) inhibiting the disease;
c) slowing or arresting the development of clinical symptoms; and/or
d) relieving the disease, that is, causing the regression of clinical symptoms.

“Patient” refers to an animal, such as a mammal, that has been or will be the object of treatment, observation or experiment. The methods of the invention can be useful in both human therapy and veterinary applications. In some embodiments, the patient is a mammal; in some embodiments the patient is human; and in some embodiments the patient is chosen from cats and dogs.

The compounds of Formula I can be named and numbered in the manner described below. For example, using nomenclature software, such as Pipeline Pilot or Nomenclator™ available from ChemInnovation Software, Inc., the compound: 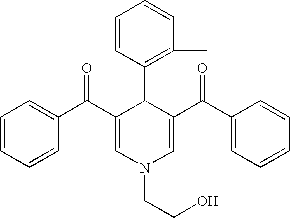
can be named 1-(2-hydroxyethyl)-4-(2-methylphenyl)-5-(phenylcarbonyl)(3-1,4-dihydropyridyl)phenyl ketone. That same compound, using the structure=name algorithm from ChemDraw 9, can be named (1-(2-hydroxyethyl)-4-o-tolyl-1,4-dihydropyridine-3,5-diyl)bis(phenylmethanone).

The present invention is directed to certain chemical entities that cause mitotic arrest and cell death. Accordingly, provided is at least one chemical entity chosen from compounds of Formula I: 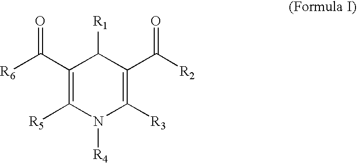
and pharmaceutically acceptable salts, solvates, chelates, non-covalent complexes, prodrugs, and mixtures thereof, wherein
R₁ is chosen from optionally substituted cycloalkyl, optionally substituted heterocycloalkyl, optionally substituted aryl and optionally substituted heteroaryl;
R₂ is chosen from optionally substituted aryl, optionally substituted heteroaryl, optionally substituted alkoxy, and optionally substituted amino;
R₃ is chosen from hydrogen and optionally substituted alkyl;
R₄ is chosen from hydrogen, optionally substituted alkyl, optionally substituted heterocycloalkyl, optionally substituted acyl, optionally substituted aryl, optionally substituted heteroaryl, aminocarbonyl, sulfonyl, optionally substituted alkoxycarbonyl, and optionally substituted cycloalkyl;
R₅ is chosen from hydrogen and optionally substituted alkyl; and
R₆ is chosen from optionally substituted aryl, optionally substituted heteroaryl, optionally substituted alkoxy, and optionally substituted amino.

In some embodiments, R₁ is chosen from optionally substituted aryl and optionally substituted heteroaryl. In some embodiments, R₁ is chosen from aryl and heteroaryl, either of which is optionally substituted with one, two, or three groups chosen from halo, optionally substituted lower alkyl, optionally substituted lower alkoxy, and hydroxy. In some embodiments, R₁ is chosen from aryl and heteroaryl, either of which is optionally substituted with one, two, or three groups chosen from halo, methyl, trifluoromethyl, ethyl, methoxy, ethoxy, and hydroxy.

In some embodiments, R₁ is chosen from optionally substituted phenyl and optionally substituted pyridinyl. In some embodiments, R₁ is chosen from phenyl and pyridinyl, either of which is optionally substituted with one, two, or three groups chosen from halo, optionally substituted lower alkyl, optionally substituted lower alkoxy, and hydroxy. In some embodiments, R₁ is chosen from phenyl and pyridinyl, either of which is optionally substituted with one, two, or three groups chosen from halo, methyl, trifluoromethyl, ethyl, methoxy, ethoxy, and hydroxy. In some embodiments, R₁ is chosen from phenyl, 2-methylphenyl, 2,3-dimethylphenyl, 3-halo-2-methylphenyl, and pyridinyl.

In some embodiments, R₂ is chosen from optionally substituted aryl and optionally substituted heteroaryl. In some embodiments, R₂ is chosen from aryl and heteroaryl, either of which is optionally substituted with one, two, or three groups chosen from halo, optionally substituted lower alkyl, optionally substituted lower alkoxy, and hydroxy. In some embodiments, R₂ is chosen from aryl and heteroaryl, either of which is which is optionally substituted with one, two, or three groups chosen from halo, methyl, trifluoromethyl, ethyl, methoxy, ethoxy, and hydroxy.

In some embodiments, R₂ is chosen from optionally substituted phenyl and optionally substituted pyridinyl. In some embodiments, R₂ is chosen from phenyl and pyridinyl, either of which is optionally substituted with one, two, or three groups chosen from halo, optionally substituted lower alkyl, optionally substituted lower alkoxy, and hydroxy. In some embodiments, R₂ is chosen from phenyl and pyridinyl, either of which is which is optionally substituted with one, two, or three groups chosen from halo, methyl, trifluoromethyl, ethyl, methoxy, ethoxy, and hydroxy. In some embodiments, R₂ is chosen from phenyl and pyridinyl.

In some embodiments, R₃ is chosen from hydrogen and optionally substituted lower alkyl. In some embodiments, R₃ is chosen from hydrogen and lower alkyl. In some embodiments, R₃ is chosen from hydrogen and methyl.

In some embodiments, R₄ is chosen from hydrogen, optionally substituted lower alkyl, optionally substituted heterocycloalkyl, and optionally substituted cycloalkyl. In some embodiments, R₄ is chosen from hydrogen, allyl, and lower alkyl optionally substituted with optionally substituted phenyl, hydroxy, lower alkoxy, alkoxycarbonyl, optionally substituted aminocarbonyl, heterocycloalkyl, acyloxy, optionally substituted amino, and carboxy. In some embodiments, R₄ is chosen from hydrogen, 2-hydroxyethyl, benzyl, 2-methoxyethyl, 2-hydroxycyclopentyl, 1,3-dihydroxypropan-2-yl, cyclopentyl, methyl, 2-morpholinoethyl, 2-methoxy-2-oxoethyl, 2-(methylamino)-2-oxoethyl, 2-acetoxyethyl, (R)-1-hydroxypropan-2-yl, (S)-1-hydroxypropan-2-yl, (R)-2-hydroxypropyl, (S)-2-hydroxypropyl, propyl, 2-(dimethylamino)ethyl, 2-(piperazin-1-yl)ethyl, 2-amino-2-oxoethyl, carboxymethyl, 3-hydroxypropyl, ethyl, 3-ethoxy-3-oxopropyl, 1,3-dihydroxypropan-2-yl, (tetrahydrofuran-2-yl)methyl, 2-ethoxy-2-oxoethyl, 3,3-dimethyl-1-(methylamino)-1-oxobutan-2-yl, 2-oxoazepan-3-yl, 1-hydroxy-3-methylbutan-2-yl, 2-(piperazin-1-yl)ethyl, 2-(4-acetylpiperazin-1-yl)ethyl, 1-propionyl, 2-(dimethylamino)-2-oxoethyl, 2-(4-(tert-butoxycarbonyl)piperazin-1-yl)-2-oxoethyl, 2-oxo-2-(piperidin-1-yl)ethyl, and 2-(2-(dimethylamino)ethylamino)-2-oxoethyl.

In some embodiments, R₅ is chosen from hydrogen and optionally substituted lower alkyl. In some embodiments, R₅ is chosen from hydrogen and lower alkyl. In some embodiments, R₅ is chosen from hydrogen and methyl.

In some embodiments, R₆ is chosen from optionally substituted aryl and optionally substituted heteroaryl. In some embodiments, R₆ is chosen from aryl and heteroaryl, either of which is optionally substituted with one, two, or three groups chosen from halo, optionally substituted lower alkyl, optionally substituted lower alkoxy, and hydroxy. In some embodiments, R₆ is chosen from aryl and heteroaryl, either of which is which is optionally substituted with one, two, or three groups chosen from halo, methyl, trifluoromethyl, ethyl, methoxy, ethoxy, and hydroxy.

In some embodiments, R₆ is chosen from optionally substituted phenyl and optionally substituted pyridinyl. In some embodiments, R₆ is chosen from phenyl and pyridinyl, either of which is optionally substituted with one, two, or three groups chosen from halo, optionally substituted lower alkyl, optionally substituted lower alkoxy, and hydroxy. In some embodiments, R₆ is chosen from phenyl and pyridinyl, either of which is which is optionally substituted with one, two, or three groups chosen from halo, methyl, trifluoromethyl, ethyl, methoxy, ethoxy, and hydroxy. In some embodiments, R₆ is chosen from phenyl and pyridinyl.

Particular compounds of Formula I are chosen from
ethyl 5-(ethoxycarbonyl)-4-phenyl-1-benzyl-1,4-dihydropyridine-3-carboxylate;
ethyl 5-(ethoxycarbonyl)-1-(2-hydroxyethyl)-4-phenyl-1,4-dihydropyridine-3-carboxylate;
4-phenyl-1-benzyl-1,4-dihydropyridine-3,5-dicarboxylic acid;
1-(2-hydroxyethyl)-4-phenyl-5-(phenylcarbonyl)(3-1,4-dihydropyridyl)phenyl ketone;
phenyl 4-phenyl-5-(phenylcarbonyl)-1-benzyl(3-1,4-dihydropyridyl)ketone;
1-(2-hydroxyethyl)-4-(2-methylphenyl)-5-(phenylcarbonyl)(3-1,4-dihydropyridyl)phenyl ketone;
1-(2-hydroxyethyl)-4-(3-methoxyphenyl)-5-(phenylcarbonyl)(3-1,4-dihydropyridyl)phenyl ketone;
4-(4-chlorophenyl)-1-(2-hydroxyethyl)-5-(phenylcarbonyl)(3-1,4-dihydropyridyl)phenyl ketone;
1-(2-hydroxyethyl)-4-(3-methylphenyl)-5-(phenylcarbonyl)(3-1,4-dihydropyridyl)phenyl ketone;
1-(2-hydroxyethyl)-4-(2-methoxyphenyl)-5-(phenylcarbonyl)(3-1,4-dihydropyridyl)phenyl ketone;
1-(2-hydroxyethyl)-4-(4-methylphenyl)-5-(phenylcarbonyl)(3-1,4-dihydropyridyl)phenyl ketone;
4-(2,3-dichlorophenyl)-1-(2-hydroxyethyl)-5-(phenylcarbonyl)(3-1,4-dihydropyridyl)phenyl ketone;
1-(2-methoxyethyl)-4-(2-methylphenyl)-5-(phenylcarbonyl)(3-1,4-dihydropyridyl)phenyl ketone;
1-(2-hydroxycyclopentyl)-4-(2-methylphenyl)-5-(phenylcarbonyl)(3-1,4-dihydropyridyl)phenyl ketone;
1-[2-hydroxy-1-(hydroxymethyl)ethyl]-4-(2-methylphenyl)-5-(phenylcarbonyl)(3-1,4-dihydropyridyl)phenyl ketone;
1-cyclopentyl-4-(2-methylphenyl)-5-(phenylcarbonyl)(3-1,4-dihydropyridyl)phenyl ketone;
1-methyl-4-(2-methylphenyl)-5-(phenylcarbonyl)(3-1,4-dihydropyridyl)phenyl ketone;
4-(2-methylphenyl)-1-(2-morpholin-4-ylethyl)-5-(phenylcarbonyl)(3-1,4-dihydropyridyl)phenyl ketone;
methyl 2-[3,5-bis(phenylcarbonyl)-4-(2-methylphenyl)-1,4-dihydropyridyl]acetate;
2-[3,5-bis(phenylcarbonyl)-4-(2-methylphenyl)(1,4-dihydropyridyl)]-N-methylacetamide;
4-(2,4-dichlorophenyl)-1-(2-hydroxyethyl)-5-(phenylcarbonyl)(3-1,4-dihydropyridyl)phenyl ketone;
4-(2,5-dichlorophenyl)-1-(2-hydroxyethyl)-5-(phenylcarbonyl)(3-1,4-dihydropyridyl)phenyl ketone;
2-[4-(2,3-dimethylphenyl)-3,5-bis(phenylcarbonyl)-1,4-dihydropyridyl]ethyl acetate;
2-[3,5-bis(phenylcarbonyl)-4-(3-methylphenyl)-1,4-dihydropyridyl]ethyl acetate;
1-((1R)-2-hydroxy-isopropyl)-4-(2-methylphenyl)-5-(phenylcarbonyl)(3-1,4-dihydropyridyl)phenyl ketone;
1-((1S)-2-hydroxy-isopropyl)-4-(2-methylphenyl)-5-(phenylcarbonyl)(3-1,4-dihydropyridyl)phenyl ketone;
1-((2R)-2-hydroxypropyl)-4-(2-methylphenyl)-5-(phenylcarbonyl)(3-1,4-dihydropyridyl)phenyl ketone;
1-((2S)-2-hydroxypropyl)-4-(2-methylphenyl)-5-(phenylcarbonyl)(3-1,4-dihydropyridyl)phenyl ketone;
4-(3,4-dichlorophenyl)-1-(2-hydroxyethyl)-5-(phenylcarbonyl)(3-1,4-dihydropyridyl)phenyl ketone;
4-(2-methylphenyl)-5-(phenylcarbonyl)-1-propyl(3-1,4-dihydropyridyl)phenyl ketone;
4-(2,3-dimethylphenyl)-1-(2-hydroxyethyl)-5-(phenylcarbonyl)(3-1,4-dihydropyridyl)phenyl ketone;
1-(2-hydroxyethyl)-4-(2-naphthyl)-5-(phenylcarbonyl)(3-1,4-dihydropyridyl)phenyl ketone;
1-[2-(dimethylamino)ethyl]-4-(2-methylphenyl)-5-(phenylcarbonyl)(3-1,4-dihydropyridyl)phenyl ketone;
4-(2-fluorophenyl)-1-(2-hydroxyethyl)-5-(phenylcarbonyl)(3-1,4-dihydropyridyl)phenyl ketone;
4-(2-chlorophenyl)-1-(2-hydroxyethyl)-5-(phenylcarbonyl)(3-1,4-dihydropyridyl)phenyl ketone;
4-(2,5-dimethylphenyl)-1-(2-hydroxyethyl)-5-(phenylcarbonyl)(3-1,4-dihydropyridyl)phenyl ketone;
4-(3,5-dichlorophenyl)-1-(2-hydroxyethyl)-5-(phenylcarbonyl)(3-1,4-dihydropyridyl)phenyl ketone;
4-(2-ethylphenyl)-1-(2-hydroxyethyl)-5-(phenylcarbonyl)(3-1,4-dihydropyridyl)phenyl ketone;
4-(2-methylphenyl)-5-(phenylcarbonyl)-1-(2-piperazinylethyl)(3-1,4-dihydropyridyl)phenyl ketone;
2-[3,5-bis(phenylcarbonyl)-4-(2-methylphenyl)-1,4-dihydropyridyl]acetamide;
2-[3,5-bis(phenylcarbonyl)-4-(2-methylphenyl)-1,4-dihydropyridyl]acetic acid;
2-[3,5-bis(phenylcarbonyl)-4-(2-methylphenyl)(1,4-dihydropyridyl)]-N,N-dimethylacetamide;
ethyl 5-(ethoxycarbonyl)-1-(2-hydroxyethyl)-4-(2-methylphenyl)-1,4-dihydropyridine-3-carboxylate;
4-cyclopropyl-1-(2-hydroxyethyl)-5-(phenylcarbonyl)(3-1,4-dihydropyridyl)phenyl ketone;
1-(3-hydroxypropyl)-4-(2-methylphenyl)-5-(phenylcarbonyl)(3-1,4-dihydropyridyl)phenyl ketone;
1-ethyl-4-(2-methylphenyl)-5-(phenylcarbonyl)(3-1,4-dihydropyridyl)phenyl ketone;
1-((1R)-2-hydroxy-isopropyl)-4-(2,3-dimethylphenyl)-5-(phenylcarbonyl)(3-1,4-dihydropyridyl)phenyl ketone;
1-((1S)-2-hydroxy-isopropyl)-4-(2,3-dimethylphenyl)-5-(phenylcarbonyl)(3-1,4-dihydropyridyl)phenyl ketone;
4-(2,3-dimethylphenyl)-5-(phenylcarbonyl)-1-propyl(3-1,4-dihydropyridyl)phenyl ketone;
4-(2,3-dimethylphenyl)-1-(2-hydroxycyclopentyl)-5-(phenylcarbonyl)(3-1,4-dihydropyridyl)phenyl ketone;
4-(2,3-dimethylphenyl)-1-cyclopentyl-5-(phenylcarbonyl)(3-1,4-dihydropyridyl)phenyl ketone;
1-(2-hydroxyethyl)-5-(phenylcarbonyl)-4-(3,3,3-trifluoropropyl)(3-1,4-dihydropyridyl)phenyl ketone;
4-(2-methylphenyl)-5-(phenylcarbonyl)(3-1,4-dihydropyridyl)phenyl ketone;
1-[5-acetyl-1-(2-hydroxyethyl)-4-(2-methylphenyl)-3-1,4-dihydropyridyl]ethan-1-one;
4-cyclohexyl-1-(2-hydroxyethyl)-5-(phenylcarbonyl)(3-1,4-dihydropyridyl)phenyl ketone;
1-(2-hydroxyethyl)-5-(phenylcarbonyl)-4-[2-(trifluoromethyl)phenyl](3-1,4-dihydropyridyl)phenyl ketone;
4-(3-chlorophenyl)-1-(2-hydroxyethyl)-5-(phenylcarbonyl)(3-1,4-dihydropyridyl)phenyl ketone;
1-(2-hydroxyethyl)-4-(4-methoxyphenyl)-5-(phenylcarbonyl)(3-1,4-dihydropyridyl)phenyl ketone;
4-(2-bromophenyl)-1-(2-hydroxyethyl)-5-(phenylcarbonyl)(3-1,4-dihydropyridyl)phenyl ketone;
4-(2-methylphenyl)-5-(phenylcarbonyl)-1-benzyl(3-1,4-dihydropyridyl)phenyl ketone;
methyl 5-(methoxycarbonyl)-2,6-dimethyl-4-(2-methylthiophenyl)-1,4-dihydropyridine-3-carboxylate;
methyl 5-(methoxycarbonyl)-2,6-dimethyl-4-(2-methylthiophenyl)pyridine-3-carboxylate;
2,6-dimethyl-4-(2-methylphenyl)-5-(phenylcarbonyl)(3-1,4-dihydropyridyl)phenyl ketone;
tert-butyl 4-{2-[3,5-bis(phenylcarbonyl)-4-(2-methylphenyl)-1,4-dihydropyridyl]acetyl}piperazinecarboxylate;
2-[3,5-bis(phenylcarbonyl)-4-(2-methylphenyl)(1,4-dihydropyridyl)]-1-piperidylethan-1-one;
N-[2-(dimethylamino)ethyl]-2-[3,5-bis(phenylcarbonyl)-4-(2-methylphenyl)(1,4-dihydropyridyl)]acetamide;
2,6-dimethyl-4-(2-methylphenyl)-5-(phenylcarbonyl)-1,4-dihydropyridine-3-carboxylic acid;
phenylmethyl 2,6-dimethyl-4-(2-methylphenyl)-5-(phenylcarbonyl)-1,4-dihydropyridine-3-carboxylate;
[2,6-dimethyl-4-(2-methylphenyl)-5-(phenylcarbonyl)(3-1,4-dihydropyridyl)]-N-benzylcarboxamide; and
2-[4-(2,3-dimethylphenyl)-3,5-bis(phenylcarbonyl)-2,6-dimethyl(1,4-dihydropyridyl)]-N-methylacetamide.

The compounds of the invention can be synthesized utilizing techniques well known in the art from commercially available starting materials and reagents. For example, the compounds of the invention can be prepared as shown below:

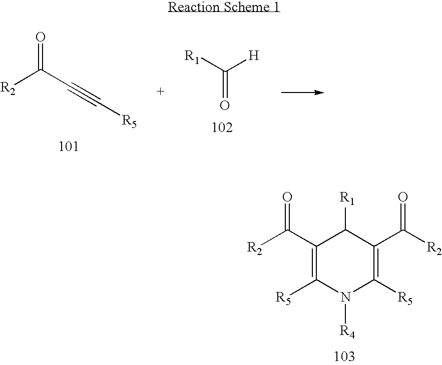

Referring to Reaction Scheme 1, to a scintillation vial is added acetic acid and a compound of Formula R₄—NH₂. After the mixture is allowed to cool to room temperature, a compound of Formula 102 and a compound of Formula 101 is added to the vial and the resulting solution is heated to about 100° C. for about 20 minutes. The product, a compound of Formula 103, is isolated and optionally purified.

In some embodiments, the chemical entities described herein are administered as a pharmaceutical composition or formulation. Accordingly, provided are pharmaceutical formulations comprising at least one chemical entity chosen from compounds of Formula 1 and pharmaceutically acceptable salts, solvates, chelates, non-covalent complexes, prodrugs, and mixtures thereof, together with at least one pharmaceutically acceptable vehicle chosen from carriers, adjuvants, and excipients.

Pharmaceutically acceptable vehicles must be of sufficiently high purity and sufficiently low toxicity to render them suitable for administration to the animal being treated. The vehicle can be inert or it can possess pharmaceutical benefits. The amount of vehicle employed in conjunction with the chemical entity is sufficient to provide a practical quantity of material for administration per unit dose of the chemical entity.

Exemplary pharmaceutically acceptable carriers or components thereof are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; synthetic oils; vegetable oils, such as peanut oil, cottonseed oil, sesame oil, olive oil, and corn oil; polyols such as propylene glycol, glycerine, sorbitol, mannitol, and polyethylene glycol; alginic acid; phosphate buffer solutions; emulsifiers, such as the TWEENS; wetting agents, such sodium lauryl sulfate; coloring agents; flavoring agents; tableting agents; stabilizers; antioxidants; preservatives; pyrogen-free water; isotonic saline; and phosphate buffer solutions.

Optional active agents may be included in a pharmaceutical composition, which do not substantially interfere with the activity of the chemical entity of the present invention.

Effective concentrations of at least one chemical entity chosen from compounds of Formula 1 and pharmaceutically acceptable salts, solvates, chelates, non-covalent complexes, prodrugs, and mixtures thereof, are mixed with a suitable pharmaceutically acceptable vehicle. In instances in which the chemical entity exhibits insufficient solubility, methods for solubilizing compounds may be used. Such methods are known to those of skill in this art, and include, but are not limited to, using cosolvents, such as dimethylsulfoxide (DMSO), using surfactants, such as TWEEN, or dissolution in aqueous sodium bicarbonate.

Upon mixing or addition of the chemical entity described herein, the resulting mixture may be a solution, suspension, emulsion or the like. The form of the resulting mixture depends upon a number of factors, including the intended mode of administration and the solubility of the chemical entity in the chosen vehicle. The effective concentration sufficient for ameliorating the symptoms of the disease treated may be empirically determined.

Chemical entities described herein may be administered orally, topically, parenterally, intravenously, by intramuscular injection, by inhalation or spray, sublingually, transdermally, via buccal administration, rectally, as an ophthalmic solution, or by other means, in dosage unit formulations.

Dosage formulations suitable for oral use, include, for example, tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents, such as sweetening agents, flavoring agents, coloring agents and preserving agents, in order to provide pharmaceutically elegant and palatable preparations. In some embodiments, oral formulations contain from 0.1 to 99% of at least one chemical entity described herein. In some embodiments, oral formulations contain at least 5% (weight %) of at least one chemical entity described herein. Some embodiments contain from 25% to 50% or from 5% to 75% of at least one chemical entity described herein.

Orally administered compositions also include liquid solutions, emulsions, suspensions, powders, granules, elixirs, tinctures, syrups, and the like. The pharmaceutically acceptable carriers suitable for preparation of such compositions are well known in the art. Oral formulations may contain preservatives, flavoring agents, sweetening agents, such as sucrose or saccharin, taste-masking agents, and coloring agents.

Typical components of carriers for syrups, elixirs, emulsions and suspensions include ethanol, glycerol, propylene glycol, polyethylene glycol, liquid sucrose, sorbitol and water. Syrups and elixirs may be formulated with sweetening agents, for example glycerol, propylene glycol, sorbitol or sucrose. Such formulations may also contain a demulcent.

Chemical entities described herein can be incorporated into oral liquid preparations such as aqueous or oily suspensions, solutions, emulsions, syrups, or elixirs, for example. Moreover, formulations containing these chemical entities can be presented as a dry product for constitution with water or other suitable vehicle before use. Such liquid preparations can contain conventional additives, such as suspending agents (e.g., sorbitol syrup, methyl cellulose, glucose/sugar, syrup, gelatin, hydroxyethyl cellulose, carboxymethyl cellulose, aluminum stearate gel, and hydrogenated edible fats), emulsifying agents (e.g., lecithin, sorbitan monsoleate, or acacia), non-aqueous vehicles, which can include edible oils (e.g., almond oil, fractionated coconut oil, silyl esters, propylene glycol and ethyl alcohol), and preservatives (e.g., methyl or propyl p-hydroxybenzoate and sorbic acid).

For a suspension, typical suspending agents include methylcellulose, sodium carboxymethyl cellulose, AVICEL RC-591, tragacanth and sodium alginate; typical wetting agents include lecithin and polysorbate 80; and typical preservatives include methyl paraben and sodium benzoate.

Aqueous suspensions contain the active material(s) in admixture with excipients suitable for the manufacture of aqueous suspensions. Such excipients are suspending agents, for example sodium carboxymethylcellulose, methylcellulose, hydropropylmethylcellulose, sodium alginate, polyvinylpyrrolidone, gum tragacanth and gum acacia; dispersing or wetting agents; may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol substitute, or condensation products of ethylene oxide with partial esters derived from fatty acids and hexitol anhydrides, for example polyethylene sorbitan substitute. The aqueous suspensions may also contain one or more preservatives, for example ethyl, or n-propyl p-hydroxybenzoate.

Oily suspensions may be formulated by suspending the active ingredients in a vegetable oil, for example peanut oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Pharmaceutical compositions of the invention may also be in the form of oil-in-water emulsions. The oily phase may be a vegetable oil, for example olive oil or peanut oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above.

Tablets typically comprise conventional pharmaceutically acceptable adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, can be useful adjuvants for chewable tablets. Capsules (including time release and sustained release formulations) typically comprise one or more solid diluents disclosed above. The selection of carrier components often depends on secondary considerations like taste, cost, and shelf stability.

Such compositions may also be coated by conventional methods, typically with pH or time-dependent coatings, such that the chemical entity is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methylcellulose phthalate, ethyl cellulose, Eudragit coatings, waxes and shellac.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be sterile injectable solution or suspension in a non-toxic parentally acceptable vehicle, for example as a solution in 1,3-butanediol. Among the acceptable vehicles that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid can be useful in the preparation of injectables.

Chemical entities described herein may be administered parenterally in a sterile medium. Parenteral administration includes subcutaneous injections, intravenous, intramuscular, intrathecal injection or infusion techniques. Chemical entities described herein, depending on the vehicle and concentration used, can either be suspended or dissolved in the vehicle. Advantageously, adjuvants such as local anesthetics, preservatives and buffering agents can be dissolved in the vehicle. In many compositions for parenteral administration the carrier comprises at least 90% by weight of the total composition. In some embodiments, the carrier for parenteral administration is chosen from propylene glycol, ethyl oleate, pyrrolidone, ethanol, and sesame oil.

Chemical entites described herein may also be administered in the form of suppositories for rectal administration of the drug. These compositions can be prepared by mixing the drug with a suitable non-irritating excipient that is solid at ordinary temperatures but liquid at rectal temperature and will therefore melt in the rectum to release the drug. Such materials include cocoa butter and polyethylene glycols.

Chemical entities described herein may be formulated for local or topical application, such as for topical application to the skin and mucous membranes, such as in the eye, in the form of gels, creams, and lotions and for application to the eye. Topical compositions may be in any form including, for example, solutions, creams, ointments, gels, lotions, milks, cleansers, moisturizers, sprays, skin patches, and the like.

Such solutions may be formulated as 0.01%-10% isotonic solutions, pH 5-7, with appropriate salts. Chemical entities described herein may also be formulated for transdermal administration as a transdermal patch.

Topical compositions comprising at least one chemical entity described herein can be admixed with a variety of carrier materials well known in the art, such as, for example, water, alcohols, aloe vera gel, allantoin, glycerine, vitamin A and E oils, mineral oil, propylene glycol, PPG-2 myristyl propionate, and the like.

Other materials suitable for use in topical carriers include, for example, emollients, solvents, humectants, thickeners and powders. Examples of each of these types of materials, which can be used singly or as mixtures of one or more materials, are as follows:

Representative emollients include stearyl alcohol, glyceryl monoricinoleate, glyceryl monostearate, propane-1,2-diol, butane-1,3-diol, mink oil, cetyl alcohol, iso-propyl isostearate, stearic acid, iso-butyl palmitate, isocetyl stearate, oleyl alcohol, isopropyl laurate, hexyl laurate, decyl oleate, octadecan-2-ol, isocetyl alcohol, cetyl palmitate, dimethylpolysiloxane, di-n-butyl sebacate, iso-propyl myristate, iso-propyl palmitate, iso-propyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, and myristyl myristate; propellants, such as propane, butane, iso-butane, dimethyl ether, carbon dioxide, and nitrous oxide; solvents, such as ethyl alcohol, methylene chloride, iso-propanol, castor oil, ethylene glycol monoethyl ether, diethylene glycol monobutyl ether, diethylene glycol monoethyl ether, dimethyl sulphoxide, dimethyl formamide, tetrahydrofuran; humectants, such as glycerin, sorbitol, sodium 2-pyrrolidone-5-carboxylate, soluble collagen, dibutyl phthalate, and gelatin; and powders, such as chalk, talc, fullers earth, kaolin, starch, gums, colloidal silicon dioxide, sodium polyacrylate, tetra alkyl ammonium smectites, trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose, and ethylene glycol monostearate.

Chemical entities described herein may also be topically administered in the form of liposome delivery systems, such as small unilamellar vesicles, large unilamellar vesicles, and multilamellar vesicles. Liposomes can be formed from a variety of phospholipids, such as cholesterol, stearylamine or phosphatidylcholines.

Other compositions useful for attaining systemic delivery of the chemical entity include sublingual, buccal and nasal dosage forms. Such compositions typically comprise one or more of soluble filler substances such as sucrose, sorbitol and mannitol, and binders such as acacia, microcrystalline cellulose, carboxymethyl cellulose, and hydroxypropyl methylcellulose. Glidants, lubricants, sweeteners, colorants, antioxidants and flavoring agents disclosed above may also be included.

Compositions for inhalation typically can be provided in the form of a solution, suspension or emulsion that can be administered as a dry powder or in the form of an aerosol using a conventional propellant (e.g., dichlorodifluoromethane or trichlorofluoromethane).

The compositions of the present invention may also optionally comprise an activity enhancer. The activity enhancer can be chosen from a wide variety of molecules that function in different ways to enhance or be independent of therapeutic effects of the chemical entities described herein. Particular classes of activity enhancers include skin penetration enhancers and absorption enhancers.

Pharmaceutical compositions of the invention may also contain additional active agents that can be chosen from a wide variety of molecules, which can function in different ways to enhance the therapeutic effects of at least one chemical entity described herein. These optional other active agents, when present, are typically employed in the compositions of the invention at a level ranging from 0.01% to 15%. Some embodiments contain from 0.1% to 10% by weight of the composition. Other embodiments contain from 0.5% to 5% by weight of the composition.

The invention includes packaged pharmaceutical formulations. Such packaged formulations include a pharmaceutical composition comprising at least one chemical entity chosen from compounds of Formula 1 and pharmaceutically acceptable salts, solvates, chelates, non-covalent complexes, prodrugs, and mixtures thereof, and instructions for using the composition to treat a mammal (typically a human patient). In some embodiments, the instructions are for using the pharmaceutical composition to treat a patient suffering from a cellular proliferation disease. The invention can include providing prescribing information; for example, to a patient or health care provider, or as a label in a packaged pharmaceutical formulation. Prescribing information may include for example efficacy, dosage and administration, contraindication and adverse reaction information pertaining to the pharmaceutical formulation.

In all of the foregoing the chemical entities can be administered alone, as mixtures, or in combination with other active agents.

The chemical entities described herein can be used to treat cellular proliferation diseases. Such diseases include, but are not limited to, cancer (further discussed below), autoimmune disease, fungal disorders, arthritis, graft rejection, inflammatory bowel disease, cellular proliferation induced after medical procedures, including, but not limited to, surgery, angioplasty, and the like. Treatment includes inhibiting cellular proliferation. It is appreciated that in some cases the cells may not be in an abnormal state and still require treatment. Thus, in some embodiments, at least one chemical entity is administered to cells or individuals afflicted or subject to impending affliction with any one of these diseases or states.

The chemical entities provided herein can be used to treat cancer including solid tumors such as skin, breast, brain, cervical carcinomas, testicular carcinomas, etc. More particularly, cancers that can be treated include, but are not limited to:
Cardiac: sarcoma (angiosarcoma, fibrosarcoma, rhabdomyosarcoma, liposarcoma), myxoma, rhabdomyoma, fibroma, lipoma and teratoma;
Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;
Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma);
Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);
Liver: hepatoma (hepatocellular carcinoma), cholangiocarcinoma, hepatoblastoma, angiosarcoma, hepatocellular adenoma, hemangioma;
Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;
Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma);
Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant tertoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma);
Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma];
Skin: malignant melanoma, basal cell carcinoma, squamous cell carcinoma, Karposi's sarcoma, moles dysplastic nevi, lipoma, angioma, dermatofibroma, keloids, psoriasis; and
Adrenal glands: neuroblastoma.
   As used herein, treatment of cancer includes treatment of cancerous cells.

The chemical entities provided herein may be demonstrated to inhibit tumor cell proliferation, cell transformation and tumorigenesis in vitro and in vivo using a variety of assays known in the art, or described herein. Such assays may use cells of a cancer cell line, or cells from a patient. Many assays well-known in the art can be used to assess such survival and/or growth; for example, cell proliferation can be assayed by measuring ³H-thymidine incorporation, by direct cell count, by detecting changes in transcription, translation or activity of known genes such as proto-oncogenes (e.g., fos, myc) or cell cycle markers (Rb, cdc2, cyclin A, D1, D2, D3, E, etc). The levels of such protein and mRNA and activity can be determined by any method well known in the art.

Cell proliferation may be measured by counting samples of a cell population over time (e.g., daily cell counts). Cells may be counted using a hemacytometer and light microscopy (e.g., HyLite hemacytometer, Hausser Scientific). Cell number may be plotted against time in order to obtain a growth curve for the population of interest. In one embodiment, cells are first mixed with the dye Trypan-blue (Sigma), such that living cells exclude the dye, and are counted as viable members of the population.

DNA content and/or mitotic index of the cells may be measured, for example, based on DNA ploidy value of the cell. For example, cells in the G1 phase of the cell cycle generally contain a 2N DNA ploidy value. Cells in which DNA has been replicated but have not progressed through mitosis (e.g., cells in S-phase) will exhibit a ploidy value higher than 2N and up to 4N DNA content. Ploidy value and cell-cycle kinetics may be further measured using propidum iodide assay (see, e.g., Turner, T., et al., 1998, Prostate 34:175-81). Alternatively, the DNA ploidy may be determined by quantitation of DNA Feulgen staining (which binds to DNA in a stoichiometric manner) on a computerized microdensitometry staining system (see, e.g., Bacus, S., 1989, Am. J. Pathol. 135:783-92). In an another embodiment, DNA content may be analyzed by preparation of a chromosomal spread (Zabalou, S., 1994, Hereditas. 120:127-40; Pardue, M. L., 1994, Meth. Cell Biol. 44:333-351).

Detection of changes in length of the cell cycle or speed of cell cycle may also be used to measure inhibition of cell proliferation by the compounds of the invention. In one embodiment the length of the cell cycle is determined by the doubling time of a population of cells (e.g., using cells contacted or not contacted with one or more compounds of the invention). In another embodiment, FACS analysis is used to analyze the phase of cell cycle progression, or purify G1, S, and G2/M fractions (see e.g., Delia, D. et al., 1997, Oncogene 14:2137-47).

Lapse of cell cycle checkpoint(s), and/or induction of cell cycle checkpoint(s), may be examined by any method known in the art. Without limitation, a cell cycle checkpoint is a mechanism which ensures that certain cellular events occur in a particular order. Checkpoint genes are defined by mutations that allow late events to occur without prior completion of an early event (Weinert, T., and Hartwell, L., 1993, Genetics 134:63-80). Induction or inhibition of cell cycle checkpoint genes may be assayed, for example, by Western blot analysis, or by immunostaining, etc. Lapse of cell cycle checkpoints may be further assessed by the progression of a cell through the checkpoint without prior occurrence of specific events (e.g., progression into mitosis without complete replication of the genomic DNA).

The chemical entities provided herein can also be demonstrated to alter cell proliferation in cultured cells in vitro using methods that are well known in the art. Specific examples of cell culture models include, but are not limited to, for lung cancer, primary rat lung tumor cells (Swafford et al., 1997, Mol. Cell. Biol. 17:1366-1374) and large-cell undifferentiated cancer cell lines (Mabry et al., 1991, Cancer Cells 3:53-58); colorectal cell lines for colon cancer (Park and Gazdar, 1996, J. Cell Biochem. Suppl. 24:131-141); multiple established cell lines for breast cancer (Hambly et al., 1997, Breast Cancer Res. Treat. 43:247-258; Gierthy et al., 1997, Chemosphere 34:1495-1505; Prasad and Church, 1997, Biochem. Biophys. Res. Commun. 232:14-19); a number of well-characterized cell models for prostate cancer (Webber et al., 1996, Prostate, Part 1, 29:386-394; Part 2, 30:58-64; and Part 3, 30-136-142; Boulikas, 1997, Anticancer Res. 17:1471-1505); for genitourinary cancers, continuous human bladder cancer cell lines (Ribeiro et al., 1997, Int. J. Radiat. Biol. 72:11-20); organ cultures of transitional cell carcinomas (Booth et al., 1997, Lab Invest. 76:843-857) and rat progression models (Vet et al., 1997, Biochim. Biophys Acta 1360:39-44); and established cell lines for leukemias and lymphomas (Drexler, 1994, Leuk. Res. 18:919-927; Tohyama, 1997, Int. J. Hematol. 65:309-317).

The chemical entities provided herein can also be demonstrated to inhibit cell growth (or mitosis) in vitro. In this embodiment, cells are contacted with one or more chemical entities provided herein, and examined for lethal phenotype.

The chemical entities provided herein can also be demonstrated to inhibit tumor formation in vivo. A vast number of animal models of hyperproliferative disorders, including tumorigenesis and metastatic spread, are known in the art (see Table 317-1, Chapter 317, “Principals of Neoplasia,” in Harrison's Principals of Internal Medicine, 13th Edition, Isselbacher et al., eds., McGraw-Hill, N.Y., p. 1814; and Lovejoy et al., 1997, J. Pathol. 181:130-135). Specific examples include for lung cancer, transplantation of tumor nodules into rats (Wang et al., 1997, Ann. Thorac. Surg. 64:216-219) or establishment of lung cancer metastases in SCID mice depleted of NK cells (Yono and Sone, 1997, Gan To Kagaku Ryoho 24:489-494); for colon cancer, colon cancer transplantation of human colon cancer cells into nude mice (Gutman and Fidler, 1995, World J. Surg. 19:226-234), the cotton top tamarin model of human ulcerative colitis (Warren, 1996, Aliment. Pharmacol. Ther. Supp 12:45-47) and mouse models with mutations of the adenomatous polyposis tumor suppressor (Polakis, 1997, Biochim. Biophys. Acta 1332:F127-F147); for breast cancer, transgenic models of breast cancer (Dankfort and Muller, 1996, Cancer Treat. Res. 83:71-88; Amundadittir et al., 1996, Breast Cancer Res. Treat. 39:119-135) and chemical induction of tumors in rats (Russo and Russo, 1996, Breast Cancer Res. Treat. 39:7-20); for prostate cancer, chemically-induced and transgenic rodent models, and human xenograft models (Royai et al., 1996, Semin. Oncol. 23:35-40); for genitourinary cancers, induced bladder neoplasm in rats and mice (Oyasu, 1995, Food Chem. Toxicol. 33:747-755) and xenografts of human transitional cell carcinomas into nude rats (Jarrett et al., 1995, J. Endourol. 9:1-7); and for hematopoietic cancers, transplanted allogenic marrow in animals (Appelbaum, 1997, Leukemia 11(Suppl. 4):S15-S17). Further, general animal models applicable to many types of cancer have been described, including, but not restricted to, the p53-deficient mouse model (Donehower, 1996, Semin. Cancer Biol. 7:269-278), the Min mouse (Shoemaker et al., 1997, Biochem. Biophys. Acta, 1332:F25-F48), and immune responses to tumors in rat (Frey, 1997, Methods, 12:173-188).

For example, chemical entities provided herein can be administered to a test animal, preferably a test animal predisposed to develop a type of tumor, and the test animal subsequently examined for a decreased incidence of tumor formation in comparison with controls not administered the compound. Alternatively, chemical entities provided herein can be administered to test animals having tumors (e.g., animals in which tumors have been induced by introduction of malignant, neoplastic, or transformed cells, or by administration of a carcinogen) and subsequently examining the tumors in the test animals for tumor regression in comparison to controls not administered the chemical entity.

Another measure of inhibition is GI₅₀, defined as the concentration of the chemical entity that results in a decrease in the rate of cell growth by fifty percent. In some embodiments, the chemical entity has a GI₅₀ of less than about 1 mM; alternatively, the chemical entity has a GI₅₀ of less than about 20 μM, less than about 10 μM, less than about 1 μM, less than about 100 nM, or less than about 10 nM. Measurement of GI₅₀ is done using a cell proliferation assay such as described herein. Chemical entities provided herein were found to inhibit cell proliferation.

In vitro potency of chemical entities provided herein can be determined, for example, by assaying human ovarian cancer cells (SKOV3) for viability following a 72-hour exposure to a 9-point dilution series of compound. Cell viability is determined by measuring the absorbance of formazon, a product formed by the bioreduction of MTS/PMS, a commercially available reagent. Each point on the dose-response curve is calculated as a percent of untreated control cells at 72 hours minus background absorption (complete growth inhibition).

Anti-proliferative compounds that have been successfully applied in the clinic to treatment of cancer (cancer chemotherapeutics) have GI₅₀'s that vary greatly. For example, in A549 cells, paclitaxel GI₅₀ is 4 nM, doxorubicin is 63 nM, 5-fluorouracil is 1 μM, and hydroxyurea is 500 μM (data provided by National Cancer Institute, Developmental Therapeutic Program, http://dtp.nci.nih.gov/). Therefore, compounds that inhibit cellular proliferation, irrespective of the concentration demonstrating inhibition, have potential clinical usefulness.

Chemical entities provided herein may be administered, for example, as a pharmaceutically acceptable composition, to a patient. Depending upon the manner of introduction, the chemical entities may be formulated in a variety of ways as discussed below. The concentration of therapeutically active chemical entity in the formation may vary from about 0.1-10 wt. %.

The chemical entity may be administered alone or in combination with other treatments, i.e., radiation, or other chemotherapeutic agents such as the taxane class of agents that appear to act on microtubule formation or the camptothecin class of topoisomerase I inhibitors. When used, other chemotherapeutic agents may be administered before, concurrently, or after administration of at least one chemical entity provided herein. In some embodiments, at least one chemical entity provided herein is co-administered with one or more other chemotherapeutic agents. By “co-administer” it is meant that at least one chemical entity provided herein is administered to a patient such that the chemical entity as well as the co-administered compound may be found in the patient's bloodstream at the same time, regardless of when the compounds are actually administered, including simultaneously.

The following examples serve to more fully describe the manner of using the above-described invention. It is understood that these examples in no way serve to limit the true scope of this invention, but rather are presented for illustrative purposes.

### EXAMPLES

### Example 1

### Phenyl-Alkynyl Ketone Procedure

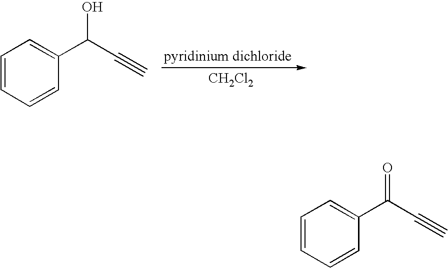

Aryl alkynyl alcohols may be prepared using the methodology of Joung, M. J.; Ahn, J. H.; Yoon, N. M. J. Org. Chem. 1996, 61(13), 4472-4475 or Mann, A.; Muller, C.; Tyrell, E. J. Chem. Soc., Perkin Trans 1 1998, (8), 1427-1438.

In a 1 L flask fitted with magnetic stirrer and 250 mL methylene chloride was added 12.25 g (56.8 mmol) pyridinium dichloride. The mixture was cooled to 0° C. and 5 g (37.9 mmol) phenyl alkynyl methanol was added dropwise via syringe. The reaction was allowed to stir for 2 hours and then purified directly through a thick plug (200 mL) of silica on a fritted funnel using methylene chloride as solvent. The solutions were concentrated with no heating to yield 3.6 g (27.7 mmol) phenyl-alkynyl ketone that was immediately place in a 0° C. freezer.
Bisbenzoyl Dihydropyridine Procedure

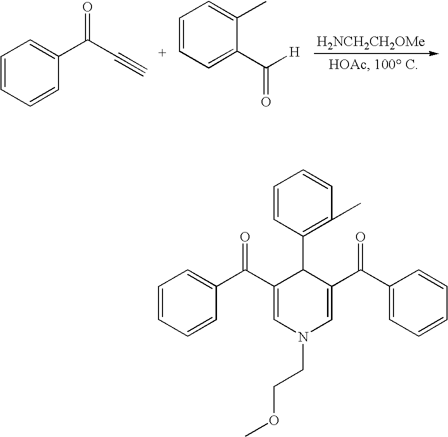

### (1-(2-methoxyethyl)-4-o-tolyl-1,4-dihydropyridine-3,5-diyl)bis(phenylmethanone)

To a 30 mL scintillation vial was added acetic acid (1 ML) and 2-methoxyethylamine (0.052 mL, 0.6 mmol). After the mixture was allowed to cool to room temperature, o-tolualdehyde (0.069 mL, 0.6 mmol) and 1-phenylprop-2-yn-1-one (0.15 g, 1.2 mmol) was added to the vial and the resulting solution was heated to 100° C. for 20 minutes. The vial was removed from heating and ice was added with stirring. Ethyl acetate was then added to the mixture, the vial shaken, and the organic layer was removed and concentrated. The resulting oil was dissolved in DMF (1.5 mL) and filtered through a fritted filter disk. The resulting solution was purified by reverse phase HPLC (30% acetonitrile starting concentration) to afford 47 mg (18%) of (1-(2-methoxyethyl)-4-o-tolyl-1,4-dihydropyridine-3,5-diyl)bis(phenylmethanone).

### Example 2

Using procedures similar to those described herein, the following compounds were prepared:
ethyl 5-(ethoxycarbonyl)-4-phenyl-1-benzyl-1,4-dihydropyridine-3-carboxylate;
ethyl 5-(ethoxycarbonyl)-1-(2-hydroxyethyl)-4-phenyl-1,4-dihydropyridine-3-carboxylate;
4-phenyl-1-benzyl-1,4-dihydropyridine-3,5-dicarboxylic acid;
1-(2-hydroxyethyl)-4-phenyl-5-(phenylcarbonyl)(3-1,4-dihydropyridyl)phenyl ketone;
phenyl 4-phenyl-5-(phenylcarbonyl)-1-benzyl(3-1,4-dihydropyridyl)ketone;
1-(2-hydroxyethyl)-4-(2-methylphenyl)-5-(phenylcarbonyl)(3-1,4-dihydropyridyl)phenyl ketone;
1-(2-hydroxyethyl)-4-(3-methoxyphenyl)-5-(phenylcarbonyl)(3-1,4-dihydropyridyl)phenyl ketone;
4-(4-chlorophenyl)-1-(2-hydroxyethyl)-5-(phenylcarbonyl)(3-1,4-dihydropyridyl)phenyl ketone;
1-(2-hydroxyethyl)-4-(3-methylphenyl)-5-(phenylcarbonyl)(3-1,4-dihydropyridyl)phenyl ketone;
1-(2-hydroxyethyl)-4-(2-methoxyphenyl)-5-(phenylcarbonyl)(3-1,4-dihydropyridyl)phenyl ketone;
1-(2-hydroxyethyl)-4-(4-methylphenyl)-5-(phenylcarbonyl)(3-1,4-dihydropyridyl)phenyl ketone;
4-(2,3-dichlorophenyl)-1-(2-hydroxyethyl)-5-(phenylcarbonyl)(3-1,4-dihydropyridyl)phenyl ketone;
1-(²-methoxyethyl)-4-(2-methylphenyl)-5-(phenylcarbonyl)(3-1,4-dihydropyridyl)phenyl ketone;
1-(2-hydroxycyclopentyl)-4-(2-methylphenyl)-5-(phenylcarbonyl)(3-1,4-dihydropyridyl)phenyl ketone;
1-[2-hydroxy-1-(hydroxymethyl)ethyl]-4-(2-methylphenyl)-5-(phenylcarbonyl)(3-1,4-dihydropyridyl)phenyl ketone;
1-cyclopentyl-4-(2-methylphenyl)-5-(phenylcarbonyl)(3-1,4-dihydropyridyl)phenyl ketone;
1-methyl-4-(2-methylphenyl)-5-(phenylcarbonyl)(3-1,4-dihydropyridyl)phenyl ketone;
4-(2-methylphenyl)-1-(2-morpholin-4-ylethyl)-5-(phenylcarbonyl)(3-1,4-dihydropyridyl)phenyl ketone;
methyl 2-[3,5-bis(phenylcarbonyl)-4-(2-methylphenyl)-1,4-dihydropyridyl]acetate;
2-[3,5-bis(phenylcarbonyl)-4-(2-methylphenyl)(1,4-dihydropyridyl)]-N-methylacetamide;
4-(2,4-dichlorophenyl)-1-(2-hydroxyethyl)-5-(phenylcarbonyl)(3-1,4-dihydropyridyl)phenyl ketone;
4-(2,5-dichlorophenyl)-1-(2-hydroxyethyl)-5-(phenylcarbonyl)(3-1,4-dihydropyridyl)phenyl ketone;
2-[4-(2,3-dimethylphenyl)-3,5-bis(phenylcarbonyl)-1,4-dihydropyridyl]ethyl acetate;
2-[3,5-bis(phenylcarbonyl)-4-(3-methylphenyl)-1,4-dihydropyridyl]ethyl acetate;
1-((1R)-2-hydroxy-isopropyl)-4-(2-methylphenyl)-5-(phenylcarbonyl)(3-1,4-dihydropyridyl)phenyl ketone;
1-((1S)-2-hydroxy-isopropyl)-4-(2-methylphenyl)-5-(phenylcarbonyl)(3-1,4-dihydropyridyl)phenyl ketone;
1-((2R)-2-hydroxypropyl)-4-(2-methylphenyl)-5-(phenylcarbonyl)(3-1,4-dihydropyridyl)phenyl ketone;
1-((2S)-2-hydroxypropyl)-4-(2-methylphenyl)-5-(phenylcarbonyl)(3-1,4-dihydropyridyl)phenyl ketone;
4-(3,4-dichlorophenyl)-1-(2-hydroxyethyl)-5-(phenylcarbonyl)(3-1,4-dihydropyridyl)phenyl ketone;
4-(2-methylphenyl)-5-(phenylcarbonyl)-1-propyl(3-1,4-dihydropyridyl)phenyl ketone;
4-(2,3-dimethylphenyl)-1-(2-hydroxyethyl)-5-(phenylcarbonyl)(3-1,4-dihydropyridyl)phenyl ketone;
1-(2-hydroxyethyl)-4-(2-naphthyl)-5-(phenylcarbonyl)(3-1,4-dihydropyridyl)phenyl ketone;
1-[2-(dimethylamino)ethyl]-4-(2-methylphenyl)-5-(phenylcarbonyl)(3-1,4-dihydropyridyl)phenyl ketone;
4-(2-fluorophenyl)-1-(2-hydroxyethyl)-5-(phenylcarbonyl)(3-1,4-dihydropyridyl)phenyl ketone;
4-(2-chlorophenyl)-1-(2-hydroxyethyl)-5-(phenylcarbonyl)(3-1,4-dihydropyridyl)phenyl ketone;
4-(2,5-dimethylphenyl)-1-(2-hydroxyethyl)-5-(phenylcarbonyl)(3-1,4-dihydropyridyl)phenyl ketone;
4-(3,5-dichlorophenyl)-1-(2-hydroxyethyl)-5-(phenylcarbonyl)(3-1,4-dihydropyridyl)phenyl ketone;
4-(2-ethylphenyl)-1-(2-hydroxyethyl)-5-(phenylcarbonyl)(3-1,4-dihydropyridyl)phenyl ketone;
4-(2-methylphenyl)-5-(phenylcarbonyl)-1-(2-piperazinylethyl)(3-1,4-dihydropyridyl)phenyl ketone;
2-[3,5-bis(phenylcarbonyl)-4-(2-methylphenyl)-1,4-dihydropyridyl]acetamide;
2-[3,5-bis(phenylcarbonyl)-4-(2-methylphenyl)-1,4-dihydropyridyl]acetic acid;
2-[3,5-bis(phenylcarbonyl)-4-(2-methylphenyl)(1,4-dihydropyridyl)]-N,N-dimethylacetamide;
ethyl 5-(ethoxycarbonyl)-1-(2-hydroxyethyl)-4-(2-methylphenyl)-1,4-dihydropyridine-3-carboxylate;
4-cyclopropyl-1-(2-hydroxyethyl)-5-(phenylcarbonyl)(3-1,4-dihydropyridyl)phenyl ketone;
1-(3-hydroxypropyl)-4-(2-methylphenyl)-5-(phenylcarbonyl)(3-1,4-dihydropyridyl)phenyl ketone;
1-ethyl-4-(2-methylphenyl)-5-(phenylcarbonyl)(3-1,4-dihydropyridyl)phenyl ketone;
1-((1R)-2-hydroxy-isopropyl)-4-(2,3-dimethylphenyl)-5-(phenylcarbonyl)(3-1,4-dihydropyridyl)phenyl ketone;
1-((1S)-2-hydroxy-isopropyl)-4-(2,3-dimethylphenyl)-5-(phenylcarbonyl)(3-1,4-dihydropyridyl)phenyl ketone;
4-(2,3-dimethylphenyl)-5-(phenylcarbonyl)-1-propyl(3-1,4-dihydropyridyl)phenyl ketone;
4-(2,3-dimethylphenyl)-1-(2-hydroxycyclopentyl)-5-(phenylcarbonyl)(3-1,4-dihydropyridyl)phenyl ketone;
4-(2,3-dimethylphenyl)-1-cyclopentyl-5-(phenylcarbonyl)(3-1,4-dihydropyridyl)phenyl ketone;
1-(2-hydroxyethyl)-5-(phenylcarbonyl)-4-(3,3,3-trifluoropropyl)(3-1,4-dihydropyridyl)phenyl ketone;
4-(2-methylphenyl)-5-(phenylcarbonyl)(3-1,4-dihydropyridyl)phenyl ketone;
1-[5-acetyl-1-(2-hydroxyethyl)-4-(2-methylphenyl)-3-1,4-dihydropyridyl]ethan-1-one;
4-cyclohexyl-1-(2-hydroxyethyl)-5-(phenylcarbonyl)(3-1,4-dihydropyridyl)phenyl ketone;
1-(2-hydroxyethyl)-5-(phenylcarbonyl)-4-[2-(trifluoromethyl)phenyl](3-1,4-dihydropyridyl)phenyl ketone;
4-(3-chlorophenyl)-1-(2-hydroxyethyl)-5-(phenylcarbonyl)(3-1,4-dihydropyridyl)phenyl ketone;
1-(2-hydroxyethyl)-4-(4-methoxyphenyl)-5-(phenylcarbonyl)(3-1,4-dihydropyridyl)phenyl ketone;
4-(2-bromophenyl)-1-(2-hydroxyethyl)-5-(phenylcarbonyl)(3-1,4-dihydropyridyl)phenyl ketone;
4-(2-methylphenyl)-5-(phenylcarbonyl)-1-benzyl(3-1,4-dihydropyridyl)phenyl ketone;
methyl 5-(methoxycarbonyl)-2,6-dimethyl-4-(2-methylthiophenyl)-1,4-dihydropyridine-3-carboxylate;
methyl 5-(methoxycarbonyl)-2,6-dimethyl-4-(2-methylthiophenyl)pyridine-3-carboxylate;
2,6-dimethyl-4-(2-methylphenyl)-5-(phenylcarbonyl)(3-1,4-dihydropyridyl)phenyl ketone;
tert-butyl 4-{2-[3,5-bis(phenylcarbonyl)-4-(2-methylphenyl)-1,4-dihydropyridyl]acetyl}piperazinecarboxylate;
2-[3,5-bis(phenylcarbonyl)-4-(2-methylphenyl)(1,4-dihydropyridyl)]-1-piperidylethan-1-one;
N-[2-(dimethylamino)ethyl]-2-[3,5-bis(phenylcarbonyl)-4-(2-methylphenyl)(1,4-dihydropyridyl)]acetamide;
2,6-dimethyl-4-(2-methylphenyl)-5-(phenylcarbonyl)-1,4-dihydropyridine-3-carboxylic acid;
phenylmethyl 2,6-dimethyl-4-(2-methylphenyl)-5-(phenylcarbonyl)-1,4-dihydropyridine-3-carboxylate;
[2,6-dimethyl-4-(2-methylphenyl)-5-(phenylcarbonyl)(3-1,4-dihydropyridyl)]-N-benzylcarboxamide; and
2-[4-(2,3-dimethylphenyl)-3,5-bis(phenylcarbonyl)-2,6-dimethyl(1,4-dihydropyridyl)]-N-methylacetamide.

### Example 3

### Cellular IC50s

In vitro potency of small molecule inhibitors is determined by assaying human ovarian cancer cells (SKOV3) for viability following a 72-hour exposure to a 10-point dilution series of compound. Cell viability is determined by measuring the absorbance of formazon, a product formed by the bioreduction of MTS/PMS, a commercially available reagent. Each point on the dose-response curve is calculated as a percent of untreated control cells at 72 hours minus background absorption (complete growth inhibition).

Materials and Solutions:

Cells: SKOV3, Ovarian Cancer (human)
Media: RPMI medium+5% Fetal Bovine Serum+2 mM L-glutamine
Colorimetric Agent for Determining Cell Viability: Promega MTS tetrazolium compound.
Control Compound for max cell kill: Topotecan, 1 uM
   Procedure:
   Day 1—Cell Plating
   1. Wash adherent SKOV3 cells in a T175 Flask with 10 mLs of PBS and add 2 mLs of 0.25% trypsin. Incubate for 5 minutes at 37° C. Rinse cells from flask using 8 mL of media (RPMI medium+5% FBS) and transfer to fresh 50 mL sterile conical. Determine cell concentration by adding 100 uL of cell suspension to 900 uL of ViaCount reagent (Guava Technology), an isotonic diluent in a micro-centrifuge tube. Place vial in Guava cell counter and set readout to acquire. Record cell count and calculate the appropriate volume of cells to achieve 300 cells/20 uL.
   2. Add 20 ul of cell suspension (300 cells/well) to all wells of 384-well CoStar plates.
   3. Incubate for 24 hours at 37° C., 100% humidity, and 5% CO₂, allowing the cells to adhere to the plates.
      Day 2—Compound Addition
   1. In a sterile 384-well CoStar assay plate, dispense 5 ul of compound at 250× highest desired concentration to wells B 11-O11 (except for H11 control well) and B14-O14 (27 compounds per plate, edge wells are not used due to evaporation). 250× compound is used to ensure final uniform concentration of vehicle (DMSO) on cells is 0.4%. Dilute 14.3 ul of 10 mM Topotecan into 10 ml of 5.8% DMSO in RPMI medium giving a final concentration of 14.3 uM stock. Add 1.5 ul of this Topotecan stock to 20 ul of cell in column 13 (rows B-O) giving a final Topotecan concentration on cells of 1 uM. ODs from these wells will be used to subtract out for background absorbance of dead cells and vehicle. Add 80 ul of medium without DMSO to each compound well in column 11 and 14. Add 40 ul medium (containing 5.8% DMSO) to all remaining wells. Serially dilute compound 2-fold from column 11 to column 2 by transferring 40 ul from one column to the next taking care to mix thoroughly each time. Similarly serially dilute compound 2-fold from column 14 to column 23.
      Final Plate Layout (27 Compounds, 10×2 Fold Dilutions, Duplicate Plates)
   2. For each compound plate, add 1.5 uL compound-containing medium in duplicate from the compound plate wells to the corresponding cell plates wells. Incubate plates for 72 hours at 37° C., 100% humidity, and 5% CO₂.
      Day 5—MTS Addition and OD Reading
   1. After 72 hours of incubation with drug, remove plates from incubator and add 4.5 ul MTS/PMS to each well. Incubate plates for 120 minutes at 37° C., 100% humidity, 5% CO₂. Read ODs at 490 nm after a 5 second shaking cycle in a 384-well spectrophotometer.
      For Data analysis, calculate normalized % of control (absorbance-background), and use XLfit to generate a dose-response curve.

The following examples serve to more fully describe the manner of using the above-described invention. It is understood that these examples in no way serve to limit the true scope of this invention, but rather are presented for illustrative purposes. All publications, including but not limited to patents and patent applications, cited in this specification are herein incorporated by reference as if each individual publication were specifically and individually indicated to be incorporated by reference herein as though fully set forth.

1. At least one chemical entity chosen from compounds of Formula I: 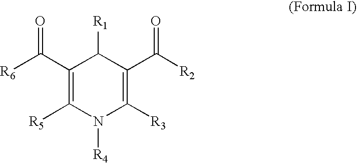
and pharmaceutically acceptable salts, solvates, chelates, non-covalent complexes, prodrugs, and mixtures thereof, wherein
R₁ is chosen from optionally substituted cycloalkyl, optionally substituted heterocycloalkyl, optionally substituted aryl and optionally substituted heteroaryl;
R₂ is chosen from optionally substituted aryl, optionally substituted heteroaryl, optionally substituted alkoxy, and optionally substituted amino;
R₃ is chosen from hydrogen and optionally substituted alkyl;
R₄ is chosen from hydrogen, optionally substituted alkyl, optionally substituted heterocycloalkyl, optionally substituted acyl, optionally substituted aryl, optionally substituted heteroaryl, aminocarbonyl, sulfonyl, optionally substituted alkoxycarbonyl, and optionally substituted cycloalkyl;
R₅ is chosen from hydrogen and optionally substituted alkyl; and
R₆ is chosen from optionally substituted aryl, optionally substituted heteroaryl, optionally substituted alkoxy, and optionally substituted amino.

2. At least one chemical entity of claim 1 wherein R₁ is chosen from optionally substituted aryl and optionally substituted heteroaryl.

3. At least one chemical entity of claim 2 wherein R₁ is chosen from aryl and heteroaryl, either of which is optionally substituted with one, two, or three groups chosen from halo, optionally substituted lower alkyl, optionally substituted lower alkoxy, and hydroxy.

4. At least one chemical entity of claim 3 wherein R₁ is chosen from aryl and heteroaryl, either of which is optionally substituted with one, two, or three groups chosen from halo, methyl, trifluoromethyl, ethyl, methoxy, ethoxy, and hydroxy.

5. At least one chemical entity of claim 2 wherein R₁ is chosen from optionally substituted phenyl and optionally substituted pyridinyl.

6. At least one chemical entity of claim 5 wherein R₁ is chosen from phenyl and pyridinyl, either of which is optionally substituted with one, two, or three groups chosen from halo, optionally substituted lower alkyl, optionally substituted lower alkoxy, and hydroxy.

7. At least one chemical entity of claim 6 wherein R₁ is chosen from phenyl and pyridinyl, either of which is optionally substituted with one, two, or three groups chosen from halo, methyl, trifluoromethyl, ethyl, methoxy, ethoxy, and hydroxy.

8. At least one chemical entity of claim 7 wherein R₁ is chosen from phenyl, 2-methylphenyl, 2,3-dimethylphenyl, 3-halo-2-methylphenyl, and pyridinyl.

9. At least one chemical entity of claim 1 wherein R₂ is chosen from optionally substituted aryl and optionally substituted heteroaryl.

10. At least one chemical entity of claim 9 wherein R₂ is chosen from aryl and heteroaryl, either of which is optionally substituted with one, two, or three groups chosen from halo, optionally substituted lower alkyl, optionally substituted lower alkoxy, and hydroxy.

11. At least one chemical entity of claim 10 wherein R₂ is chosen from aryl and heteroaryl, either of which is optionally substituted with one, two, or three groups chosen from halo, methyl, trifluoromethyl, ethyl, methoxy, ethoxy, and hydroxy.

12. At least one chemical entity of claim 9 wherein R₂ is chosen from optionally substituted phenyl and optionally substituted pyridinyl.

13. At least one chemical entity of claim 12 wherein R₂ is chosen from phenyl and pyridinyl, either of which optionally substituted with one, two, or three groups chosen from halo, optionally substituted lower alkyl, optionally substituted lower alkoxy, and hydroxy.

14. At least one chemical entity of claim 13 wherein R₂ is chosen from phenyl and pyridinyl, either of which is optionally substituted with one, two, or three groups chosen from halo, methyl, trifluoromethyl, ethyl, methoxy, ethoxy, and hydroxy.

15. At least one chemical entity of claim 14 wherein R₂ is chosen from phenyl and pyridinyl.

16. At least one chemical entity of claim 1 wherein R₃ is chosen from hydrogen and optionally substituted lower alkyl.

17. At least one chemical entity of claim 16 wherein R₃ is chosen from hydrogen and lower alkyl.

18. At least one chemical entity of claim 17 wherein R₃ is chosen from hydrogen and methyl.

19. At least one chemical entity of claim 1 wherein R₄ is chosen from hydrogen, optionally substituted lower alkyl, optionally substituted heterocycloalkyl, and optionally substituted cycloalkyl.

20. At least one chemical entity of claim 19 wherein R₄ is chosen from hydrogen, allyl, and lower alkyl optionally substituted with optionally substituted phenyl, hydroxy, lower alkoxy, alkoxycarbonyl, optionally substituted aminocarbonyl, heterocycloalkyl, acyloxy, optionally substituted amino, and carboxy.

21. At least one chemical entity of claim 20 wherein R₄ is chosen from hydrogen, 2-hydroxyethyl, benzyl, 2-methoxyethyl, 2-hydroxycyclopentyl, 1,3-dihydroxypropan-2-yl, cyclopentyl, methyl, 2-morpholinoethyl, 2-methoxy-2-oxoethyl, 2-(methylamino)-2-oxoethyl, 2-acetoxyethyl, (R)-1-hydroxypropan-2-yl, (S)-1-hydroxypropan-2-yl, (R)-2-hydroxypropyl, (S)-2-hydroxypropyl, propyl, 2-(dimethylamino)ethyl, 2-(piperazin-1-yl)ethyl, 2-amino-2-oxoethyl, carboxymethyl, 3-hydroxypropyl, ethyl, 3-ethoxy-3-oxopropyl, 1,3-dihydroxypropan-2-yl, (tetrahydrofuran-2-yl)methyl, 2-ethoxy-2-oxoethyl, 3,3-dimethyl-1-(methylamino)-1-oxobutan-2-yl, 2-oxoazepan-3-yl, 1-hydroxy-3-methylbutan-2-yl, 2-(piperazin-1-yl)ethyl, 2-(4-acetylpiperazin-1-yl)ethyl, 1-propionyl, 2-(dimethylamino)-2-oxoethyl, 2-(4-(tert-butoxycarbonyl)piperazin-1-yl)-2-oxoethyl, 2-oxo-2-(piperidin-1-yl)ethyl, and 2-(2-(dimethylamino)ethylamino)-2-oxoethyl.

22. At least one chemical entity of claim 1 wherein R₅ is chosen from hydrogen and optionally substituted lower alkyl.

23. At least one chemical entity of claim 22 wherein R₅ is chosen from hydrogen and lower alkyl.

24. At least one chemical entity of claim 23 wherein R₅ is chosen from hydrogen and methyl.

25. At least one chemical entity of claim 1 wherein R₆ is chosen from optionally substituted aryl and optionally substituted heteroaryl.

26. At least one chemical entity of claim 25 wherein R₆ is chosen from aryl and heteroaryl, either of which is optionally substituted with one, two, or three groups chosen from halo, optionally substituted lower alkyl, optionally substituted lower alkoxy, and hydroxy.

27. At least one chemical entity of claim 26 wherein R₆ is chosen from aryl and heteroaryl, either of which is optionally substituted with one, two, or three groups chosen from halo, methyl, trifluoromethyl, ethyl, methoxy, ethoxy, and hydroxy.

28. At least one chemical entity of claim 27 wherein R₆ is chosen from optionally substituted phenyl and optionally substituted pyridinyl.

29. At least one chemical entity of claim 25 wherein R₆ is chosen from phenyl and pyridinyl, either of which optionally substituted with one, two, or three groups chosen from halo, optionally substituted lower alkyl, optionally substituted lower alkoxy, and hydroxy.

30. At least one chemical entity of claim 29 wherein R₆ is chosen from phenyl and pyridinyl, either of which is optionally substituted with one, two, or three groups chosen from halo, methyl, trifluoromethyl, ethyl, methoxy, ethoxy, and hydroxy.

31. At least one chemical entity of claim 30 wherein R₆ is chosen from phenyl and pyridinyl.

32. At least one chemical entity of claim 1 wherein the compound of Formula I is chosen from:
ethyl 5-(ethoxycarbonyl)-4-phenyl-1-benzyl-1,4-dihydropyridine-3-carboxylate;
ethyl 5-(ethoxycarbonyl)-1-(2-hydroxyethyl)-4-phenyl-1,4-dihydropyridine-3-carboxylate;
4-phenyl-1-benzyl-1,4-dihydropyridine-3,5-dicarboxylic acid;
1-(2-hydroxyethyl)-4-phenyl-5-(phenylcarbonyl)(3-1,4-dihydropyridyl)phenyl ketone;
phenyl 4-phenyl-5-(phenylcarbonyl)-1-benzyl(3-1,4-dihydropyridyl)ketone;
1-(2-hydroxyethyl)-4-(2-methylphenyl)-5-(phenylcarbonyl)(3-1,4-dihydropyridyl)phenyl ketone;
1-(2-hydroxyethyl)-4-(3-methoxyphenyl)-5-(phenylcarbonyl)(3-1,4-dihydropyridyl)phenyl ketone;
4-(4-chlorophenyl)-1-(2-hydroxyethyl)-5-(phenylcarbonyl)(3-1,4-dihydropyridyl)phenyl ketone;
1-(2-hydroxyethyl)-4-(3-methylphenyl)-5-(phenylcarbonyl)(3-1,4-dihydropyridyl)phenyl ketone;
1-(2-hydroxyethyl)-4-(2-methoxyphenyl)-5-(phenylcarbonyl)(3-1,4-dihydropyridyl)phenyl ketone;
1-(2-hydroxyethyl)-4-(4-methylphenyl)-5-(phenylcarbonyl)(3-1,4-dihydropyridyl)phenyl ketone;
4-(2,3-dichlorophenyl)-1-(2-hydroxyethyl)-5-(phenylcarbonyl)(3-1,4-dihydropyridyl)phenyl ketone;
1-(2-methoxyethyl)-4-(2-methylphenyl)-5-(phenylcarbonyl)(3-1,4-dihydropyridyl)phenyl ketone;
1-(2-hydroxycyclopentyl)-4-(2-methylphenyl)-5-(phenylcarbonyl)(3-1,4-dihydropyridyl)phenyl ketone;
1-[2-hydroxy-1-(hydroxymethyl)ethyl]-4-(2-methylphenyl)-5-(phenylcarbonyl)(3-1,4-dihydropyridyl)phenyl ketone;
1-cyclopentyl-4-(2-methylphenyl)-5-(phenylcarbonyl)(3-1,4-dihydropyridyl)phenyl ketone;
1-methyl-4-(2-methylphenyl)-5-(phenylcarbonyl)(3-1,4-dihydropyridyl)phenyl ketone;
4-(2-methylphenyl)-1-(2-morpholin-4-ylethyl)-5-(phenylcarbonyl)(3-1,4-dihydropyridyl)phenyl ketone;
methyl 2-[3,5-bis(phenylcarbonyl)-4-(2-methylphenyl)-1,4-dihydropyridyl]acetate;
2-[3,5-bis(phenylcarbonyl)-4-(2-methylphenyl)(1,4-dihydropyridyl)]-N-methylacetamide;
4-(2,4-dichlorophenyl)-1-(2-hydroxyethyl)-5-(phenylcarbonyl)(3-1,4-dihydropyridyl)phenyl ketone;
4-(2,5-dichlorophenyl)-1-(2-hydroxyethyl)-5-(phenylcarbonyl)(3-1,4-dihydropyridyl)phenyl ketone;
2-[4-(2,3-dimethylphenyl)-3,5-bis(phenylcarbonyl)-1,4-dihydropyridyl]ethyl acetate;
2-[3,5-bis(phenylcarbonyl)-4-(3-methylphenyl)-1,4-dihydropyridyl]ethyl acetate;
1-((1R)-2-hydroxy-isopropyl)-4-(2-methylphenyl)-5-(phenylcarbonyl)(3-1,4-dihydropyridyl)phenyl ketone;
1-((1S)-2-hydroxy-isopropyl)-4-(2-methylphenyl)-5-(phenylcarbonyl)(3-1,4-dihydropyridyl)phenyl ketone;
1-((2R)-2-hydroxypropyl)-4-(2-methylphenyl)-5-(phenylcarbonyl)(3-1,4-dihydropyridyl)phenyl ketone;
1-((2S)-2-hydroxypropyl)-4-(2-methylphenyl)-5-(phenylcarbonyl)(3-1,4-dihydropyridyl)phenyl ketone;
4-(3,4-dichlorophenyl)-1-(2-hydroxyethyl)-5-(phenylcarbonyl)(3-1,4-dihydropyridyl)phenyl ketone;
4-(2-methylphenyl)-5-(phenylcarbonyl)-1-propyl(3-1,4-dihydropyridyl)phenyl ketone;
4-(2,3-dimethylphenyl)-1-(2-hydroxyethyl)-5-(phenylcarbonyl)(3-1,4-dihydropyridyl)phenyl ketone;
1-(2-hydroxyethyl)-4-(2-naphthyl)-5-(phenylcarbonyl)(3-1,4-dihydropyridyl)phenyl ketone;
1-[2-(dimethylamino)ethyl]-4-(2-methylphenyl)-5-(phenylcarbonyl)(3-1,4-dihydropyridyl)phenyl ketone;
4-(2-fluorophenyl)-1-(2-hydroxyethyl)-5-(phenylcarbonyl)(3-1,4-dihydropyridyl)phenyl ketone;
4-(2-chlorophenyl)-1-(2-hydroxyethyl)-5-(phenylcarbonyl)(3-1,4-dihydropyridyl)phenyl ketone;
4-(2,5-dimethylphenyl)-1-(2-hydroxyethyl)-5-(phenylcarbonyl)(3-1,4-dihydropyridyl)phenyl ketone;
4-(3,5-dichlorophenyl)-1-(2-hydroxyethyl)-5-(phenylcarbonyl)(3-1,4-dihydropyridyl)phenyl ketone;
4-(2-ethylphenyl)-1-(2-hydroxyethyl)-5-(phenylcarbonyl)(3-1,4-dihydropyridyl)phenyl ketone;
4-(2-methylphenyl)-5-(phenylcarbonyl)-1-(2-piperazinylethyl)(3-1,4-dihydropyridyl)phenyl ketone;
2-[3,5-bis(phenylcarbonyl)-4-(2-methylphenyl)-1,4-dihydropyridyl]acetamide;
2-[3,5-bis(phenylcarbonyl)-4-(2-methylphenyl)-1,4-dihydropyridyl]acetic acid;
2-[3,5-bis(phenylcarbonyl)-4-(2-methyl phenyl)(1,4-dihydropyridyl)]-N,N-dimethylacetamide;
ethyl 5-(ethoxycarbonyl)-1-(2-hydroxyethyl)-4-(2-methylphenyl)-1,4-dihydropyridine-3-carboxylate;
4-cyclopropyl-1-(2-hydroxyethyl)-5-(phenylcarbonyl)(3-1,4-dihydropyridyl)phenyl ketone;
1-(3-hydroxypropyl)-4-(2-methylphenyl)-5-(phenylcarbonyl)(3-1,4-dihydropyridyl)phenyl ketone;
1-ethyl-4-(2-methylphenyl)-5-(phenylcarbonyl)(3-1,4-dihydropyridyl)phenyl ketone;
1-((1R)-2-hydroxy-isopropyl)-4-(2,3-dimethylphenyl)-5-(phenylcarbonyl)(3-1,4-dihydropyridyl)phenyl ketone;
1-((1S)-2-hydroxy-isopropyl)-4-(2,3-dimethylphenyl)-5-(phenylcarbonyl)(3-1,4-dihydropyridyl)phenyl ketone;
4-(2,3-dimethylphenyl)-5-(phenylcarbonyl)-1-propyl(3-1,4-dihydropyridyl)phenyl ketone;
4-(2,3-dimethylphenyl)-1-(2-hydroxycyclopentyl)-5-(phenylcarbonyl)(3-1,4-dihydropyridyl)phenyl ketone;
4-(2,3-dimethylphenyl)-1-cyclopentyl-5-(phenylcarbonyl)(3-1,4-dihydropyridyl)phenyl ketone;
1-(2-hydroxyethyl)-5-(phenylcarbonyl)-4-(3,3,3-trifluoropropyl)(3-1,4-dihydropyridyl)phenyl ketone;
4-(2-methylphenyl)-5-(phenylcarbonyl)(3-1,4-dihydropyridyl)phenyl ketone;
1-[5-acetyl-1-(2-hydroxyethyl)-4-(2-methylphenyl)-3-1,4-dihydropyridyl]ethan-1-one;
4-cyclohexyl-1-(2-hydroxyethyl)-5-(phenylcarbonyl)(3-1,4-dihydropyridyl)phenyl ketone;
1-(2-hydroxyethyl)-5-(phenylcarbonyl)-4-[2-(trifluoromethyl)phenyl](3-1,4-dihydropyridyl)phenyl ketone;
4-(3-chlorophenyl)-1-(2-hydroxyethyl)-5-(phenylcarbonyl)(3-1,4-dihydropyridyl)phenyl ketone;
1-(2-hydroxyethyl)-4-(4-methoxyphenyl)-5-(phenylcarbonyl)(3-1,4-dihydropyridyl)phenyl ketone;
4-(2-bromophenyl)-1-(2-hydroxyethyl)-5-(phenylcarbonyl)(3-1,4-dihydropyridyl)phenyl ketone;
4-(2-methylphenyl)-5-(phenylcarbonyl)-1-benzyl(3-1,4-dihydropyridyl)phenyl ketone;
methyl 5-(methoxycarbonyl)-2,6-dimethyl-4-(2-methylthiophenyl)-1,4-dihydropyridine-3-carboxylate;
methyl 5-(methoxycarbonyl)-2,6-dimethyl-4-(2-methylthiophenyl)pyridine-3-carboxylate;
2,6-dimethyl-4-(2-methylphenyl)-5-(phenylcarbonyl)(3-1,4-dihydropyridyl)phenyl ketone;
tert-butyl 4-{2-[3,5-bis(phenylcarbonyl)-4-(2-methylphenyl)-1,4-dihydropyridyl]acetyl}piperazinecarboxylate;
2-[3,5-bis(phenylcarbonyl)-4-(2-methylphenyl)(1,4-dihydropyridyl)]-1-piperidylethan-1-one;
N-[2-(dimethylamino)ethyl]-2-[3,5-bis(phenylcarbonyl)-4-(2-methylphenyl)(1,4-dihydropyridyl)]acetamide;
2,6-dimethyl-4-(2-methylphenyl)-5-(phenylcarbonyl)-1,4-dihydropyridine-3-carboxylic acid;
phenylmethyl 2,6-dimethyl-4-(2-methylphenyl)-5-(phenylcarbonyl)-1,4-dihydropyridine-3-carboxylate;
[2,6-dimethyl-4-(2-methylphenyl)-5-(phenylcarbonyl)(3-1,4-dihydropyridyl)]-N-benzylcarboxamide; and
2-[4-(2,3-dimethylphenyl)-3,5-bis(phenylcarbonyl)-2,6-dimethyl(1,4-dihydropyridyl)]-N-methylacetamide.

33. A pharmaceutical composition comprising a therapeutically effective amount of at least one chemical entity of claim 1 together with at least one pharmaceutically acceptable vehicle chosen from carriers, adjuvants, and excipients.

34. A pharmaceutical composition of claim 33 wherein the composition is formulated in a form chosen from injectable fluids, aerosols, creams, gels, tablets, pills, capsules, syrups, ophthalmic solutions, and transdermal patches.

35. A packaged pharmaceutical composition, comprising
a pharmaceutical composition of claim 33; and
instructions for using the composition to treat a patient suffering from a cellular proliferative disease.

36. The packaged pharmaceutical composition of claim 35 wherein the cellular proliferative disease is cancer.

37. A method for treating a patient having a cellular proliferative disease, comprising administering to the patient an effective amount of at least one chemical entity of any claim 1.

38. The method of claim 37 wherein the cellular proliferative disease is chosen from cancer.

39. The method of claim 37 further comprising administering a chemotherapeutic agent.
